(19) United States
(12) Patent Application Publication
(10) Pub. No.: US 2010/0311123 A1
(43) Pub. Date: Dec. 9, 2010

(54) VECTOR FOR EXPRESSING ANTIBODY FRAGMENTS AND A METHOD FOR PRODUCING RECOMBINANT PHAGE THAT DISPLAYS ANTIBODY FRAGMENTS BY USING THE VECTOR

(75) Inventor: Sang-Hoon Cha, Gangwon-Do (KR)

Correspondence Address: Edwards Angell Palmer & Dodge LLP, P.O. Box 55874, Boston, MA, 02205, US

(73) Assignee: IG THERAPY, Chuncheon, Gangwon-Do (KR)

(21) Appl. No.: 12/682,880

(22) PCT Filed: Sep. 4, 2008
(86) PCT No.: PCT/KR08/05238

§ 371 Date: Jul. 19, 2010

(30) Foreign Application Priority Data
Oct. 15, 2007 (KR) 10-2007-0103483

(51) Int. Cl. C12N 15/70; C12P 21/00; C12N 7/00

(52) U.S. Cl. 435/69.6; 435/471; 435/235.1

(57) ABSTRACT

Disclosed are a plasmid vector (pLA-1 or pLT-2) for producing water-soluble light chain antibody fragments (VL+CL), a phagemid vector (pHf1g3T-1 or pHf1g3A-2) having a heavy chain antibody fragments (VH+CH1)-ΔpIII fusion protein expression and genotype-phenotype linkage function, a host transformed using the vectors, and a method of producing and selecting a water-soluble antibody and recombinant phage displaying an antibody from the host. Also, provided are a method of producing a combinatorial phage display combinatorial Fab fragment libraries DVFAB-IL and DVFAB-13 IL by using a dual vector system (DVS-II) and a method of selecting an antigen-specific human Fab fragment from the combinatorial Fab fragment libraries.

### TECHNICAL FIELD

The present invention relates to a method of constructing a plasmid vector (pLA-1 or pLT-2) for producing water-soluble light chain antibody fragments (VL+CL) and a phagemid vector (pHf1g3T-1 or pHf1g3A-2) having a heavy chain antibody fragments (VH+CH1)-ΔpIII fusion protein expression and genotype-phenotype linkage function, producing a water-soluble antibody and recombinant phage displaying an antibody from a host transformed using the vectors, and selecting an antigen-specific antibody.

Also, the present invention relates to a method of producing a combinatorial phage display Fab fragment library (DVFAB-1L) and a combinatorial Fab fragment library (DVFAB-131L) including a combination of 1 to 131 human kappa light and heavy chain repertoires by using a dual vector system (DVS-II) to introduce pLT-2 plasmid and pHf1g3A-2 phagemid into E. coli TG1 host cells, and a method of selecting an antigen-specific human Fab fragment from the combinatorial Fab fragment libraries.

### BACKGROUND ART

Phage display technology, which was first developed by the UK Medical Research Council in 1990, is technology for selecting antibody clones for a specific antigen by preparing a human antibody library and expressing it in the form of antibody fragments (Fab, ScFv) on the surface of a bacteriophage.

In producing recombinant human antibodies, the importance of the phage display technology is already well recognized (References: Clackson, T., Hoogenboom, H. R., Grifiths, A. D., Winter, G., 1991, Making antibody fragments using phage display libraries, Nature 352, 624; Hoogenboom, H., Charmes, P., 2000, Natural and designer binding sites made by phage display technology, Immunol. Today 21, 371; Hoet, R. M., Cohen, E. H., Kent, R. B., Rookey, K., Schoonbroodt, S., Hogan, S., Rem, L., Frans, N., Daukandt, M., Pieters, H., van Hegelsom, R., Neer, N. C., Nastri, H. G., Rondon, I. J., Leeds, J. A., Hufton, S. E., Huang, L., Kashin, I., Devlin, M., Kuang, G., Steukers, M., Viswanathan, M., Nixon, A E., Sexton, D. J., Hoogenboom, H. R., Ladner, R. C., 2005, Generation of high-affinity human antibodies by combining donor-derived and synthetic complementarity-determining-region diversity, Nat. Biotechnol. 23(3), 344), and a possibility of selecting almost all kinds of recombinant human monoclonal antibodies specifically reacting with antigens from a single pot antibody library system has been proposed (References: Nissim, A., et al., 1994, Antibody fragments from a ‘single pot’ phage display library as immunological reagents, EMBO J. 13, 692; Griffiths, A. D., Williams, S. C., Hartley, O., Tomlinson, I. M., Waterhouse, P., Crosby, W. L., Kontermann, R. E., Jones, P. T., Low, N. M., Allison, T. J., Prospero, T. D., Hoogenbocrn, H. R., Nissim, A., Cox, J. P. L., Harrison, J. L., Zaccolo, M., Gherardi, E., Winter, G., 1994, Isolation of high affinity htrnan antibodies directly from large synthetic repertoires, EMBO J. 13(14), 3245). This means that various antibody fragments (in the form of scFv or Fab) applicable to in vivo diagnosis and therapy may be obtained when the phage display technology is utilized (References: McCafferty, J., Griffiths, A. D., Winter, G., Chiswell, D. J., 1990, Phage antibodies: filamentous phage displaying antibody variable domains, Nature 348, 552; Winter, G., Griffiths, A. D., Hawkins, R. E., Hoogenboom, H. R., 1994, Making antibodies by phage display technology, Annu. Rev. Immunol. 12, 433; Griffiths, A. D., Duncan, A. R., 1998, Strategies for selection of antibodies by phage display, Curr. Opin. Biotechnol. 9, 102). However, there are still many technical problems in the phage display antibody technology, and thus the above-mentioned ideal antibody engineering technology is not yet realized (References: Knappik, A., Plukthun, A., 1995, Engineered turns of a recombinant antibody improve its in vivo folding, Protein Eng. 8, 81; McCafferty, J., 1996, Phage display: factors affecting panning efficiency. In: Kay, B. K., Winter, J., McCafferty, J. (Eds.), Phage Display of Peptides and Proteins, a Laboratory Manual, Academic Press, San Diego, p. 261; Krebber, A., Burmester, J., Pluckthun, A., 1996, Inclusion of an upstream transcriptional terminator in phage display vectors abolishes background expression of toxic fusions with coat protein g3p, Gene. 178, 71; Assazy, H. M. E., Highsmith, W. E., 2002, Phage display technology: clinical applications and recent innovations, Clin. Biochem. 35, 425; Baek, H., Suk, K. H., Kim, Y. H., Cha, S., 2002, An improved helper phage system for efficient isolation of specific antibody molecules in phage display, Nucleic Acids Res. 30(5), e18; Corisdeo, S., Wang, B., 2004, Functional expression and display of an antibody Fab fragment in Escherichia coli: study of vector designs and culture conditions, Protein Expr. Purif. 34, 270). That is, although such technology has an advantage in that an antigen-specific monoclonal antibody may be isolated from a “single pot” library in only a few weeks, it has also a disadvantage in that the affinity of an isolated antibody is not so high. To remedy this advantage, an in vitro affinity maturation procedure is considered in which residues of CDRs and FRs of selected antibody clones are mutated, and then higher affinity human antibody clones are selected again using a phage display method.

One of determinative factors affecting the quality of an antibody library is diversity of antibody genes inserted into phagemid (Reference: McCafferty, J., 1996, Phage display: factors affecting panning efficiency. In: Kay, B. K., Winter, J., McCafferty, J. (Eds.), Phage Display of Peptides and Proteins, a Laboratory Manual, Academic Press, San Diego, p. 261). It can be guessed that the larger the number of clones existing in an antibody library, the more the diversity of the library, but it is almost impossible to define the minimum number of clones within a library, which are required to always successfully select a gene recombinant antibody specifically binding to a specific antigen or peptide from antibody library. On the assumption that the antibody diversity of a living mouse is about 5×10⁸, it has been proposed that the size of an antibody library must be much larger than 5×10⁸ in order to secure an antibody with desired affinity and catalysis from the antibody library (Reference: Ostermeier, M., Benkovic, S. J., 2000, A two-phagemid system for the creation of non-phage displayed antibody libraries approaching one trillion members, J. Immunol. Methods, 237(1-2), 175), and indeed, only a low affinity antibody (10⁻⁶ to 10⁻⁷M) could be selected from an antibody library having a diversity of about 5×10⁸ because an in vitro system totally lacks an affinity maturation mechanism (Reference: de Bruin, R., Spelt, K., Mol, J., Koes R., Quattrocchio, F., 1999, Selection of high-affinity phage antibodies from phage display libraries, Nat. Biotechnol. 17(4), 397). In addition, it has been proposed that, due to other experimental problems, the diversity of an antibody library must be higher than 10¹⁰ in order to obtain a high affinity antibody (10⁻⁹ to 10¹⁰M) (References: Griffiths, A. D., Williams, S. C., Hartley, O., Tomlinson, I. M., Waterhouse, P., Crosby, W. L., Kontermann, R. E., Jones, P. T., Low, N. M., Allison, T. J., Prospero, T. D., Hoogenbocm, H. R., Nissim, A., Cox, J. P. L., Harrison, J. L., Zaccolo, M., Gherardi, E., Winter, G., 1994, Isolation of high affinity human antibodies directly from large synthetic repertoires, EMBO J. 13(14), 3245; Sheets, M. D., Amersdorfer, P., Finnern, R., Sargent, P., Lindquist, E., Schier, R., Hemingsen, G., Wong, C., Gerhart, J. C., Marks, J. D., Lindqvist, E., 1998, Efficient construction of a large nonimmune phage antibody library: the production of high-affinity human single-chain antibodies to protein antigens, Proc. Natl. Acad. Sci. USA. 95(11), 6157; Vaughan, T. J., Williams, A. J., Pritchard, K., Osbourn, J. K., Pope, A. R., Earnshaw, J. C., McCafferty, J., Hodits, R. A., Wilton, J., Johnson, K. S., 1996, Hunan antibodies with sub-nanomolar affinities isolated from a large non-immunized phage display library, Nat. Biotechnol. 14(3), 309). Unfortunately, however, when genes with vector DNA and antibody DNA ligated thereto are introduced into Escherichia coli cells by using electroporation, producing an antibody library having a diversity of about 10¹⁰ is a very difficult and time-consuming work due to the low transformation efficiency of E. coli.

To avoid such a technical difficulty, using a lambda phage att recombination site and Int recombinant enzyme system (Reference: Geoffroy, F., Sodoyer, R., Aujame, L., 1994, A new phage display system to construct multicombinatorial libraries of very large antibody repertoires, Gene 151, 109) or loxP site and phage P1 Cre recombinant enzyme system (References: Waterhouse, P., Griffiths, A. D., Johnson, K. S., Winter, G., 1993, Combinatorial infection and in vivo recombination: a strategy for making large phage antibody repertoires, Nucleic Acids Res. 21, 2265; Griffiths, A. D., Williams, S. C., Hartley, O., Tomlinson, I. M., Waterhouse, P., Crosby, W. L., Kontermann, R. E., Jones, P. T., Low, N. M., Allison, T. J., Prospero, T. D., Hoogenboom, H. R., Nissim, A., Cox, J. P. L., Harrison, J. L., Zaccolo, M., Gherardi, E., Winter, G., 1994, Isolation of high affinity human antibodies directly from large synthetic repertoires, EMBO J. 13(14), 3245; Tsurushita, N., Fu, H., Warren, C., 1996, Phage display vectors for in vivo recombination of immunoglobulin heavy and light chain genes to make large combinatorial libraries. Gene. 172, 59), an attempt has been made to provide an in vivo combination of heavy and light chain genes that are encoded by plasmid and phage vectors respectively in E. coli, but it may be difficult to verify the actual diversity of an antibody library produced by such a method.

Also, in order to avoid the low E. coli transformation efficiency of a DNA vector in producing an antibody library, an attempt has been made to apply a method of introducing DNA into host cells through phage infection together with a two-vector system to combinatorial antibody library production. For example, Hoogenboom et al. showed that a Fab fragment library may be produced by a two-vector system using phage vector fd-tet-DOG1 and phagemid vector pHEN1 that can be appropriately maintained in the same host cells to express functional Fab fragment molecules (Reference: Hoogenboom, H. R., Griffiths, A. D., Johnson, K. S., Chiswell, D. J., Hudson, P., Winter, G., 1991, Multi-subunit proteins on the surface of filamentous phage: methodologies for displaying antibody (Fab) heavy and light chains, Nucleic Acids Res. 19(15), 4133). However, this method may display functional Fab molecules on the surface of phage, but it is impractical to use the method for antibody library production. This is because not only recombinant fd-tet-DOG1 phage but also phage progenies obtained by infecting TG1 cells, into which phagemid vector pHEN1 is inserted, with the recombinant fd-tet-DOG1 phage have a very limited host cell infection rate. As already indicated in the M13δg3 system (References: Rakonjac, J., Jovanovic, G., Model, P., 1993, Filamentous phage infection-mediated gene expression: construction and propagation of the gIII deletion mutant helper phage R408d3, Gene. 1%, 99; McCafferty, J., 1996, Phage display: factors affecting panning efficiency. In: Kay, B. K., Winter, J., McCafferty, J. (Eds.), Phage Display of Peptides and Proteins, a Laboratory Manual, Academic Press, San Diego, p. 261), such a loss in infection function is incurred because the above recombinant phage has no wild-type g3p that interacts with sex pili of host bacteria.

Another two-vector system proposed by Ostermeier and Benkovic (Reference: Ostermeier, M., Benkovic, S. J., 2000, A two-phagemid system for the creation of non-phage displayed antibody libraries approaching one trillion members, J. Immunol. Methods, 237(1-2), 175) has a more serious problem. More specially, this two-vector system produces a combinatorial Fab library by producing heavy and light chain gene libraries in two phagemid vectors respectively, and then using the VCSM13 helper phage to produce recombinant phage having the phagemid genome and simultaneously infect bacteria host cells with the so-produced phage. However, a library produced in this way is of little value when applied to phage display because not only a problem of serious helper phage promiscuity is expected, but also a technical strategy for target-specific phage selection cannot be provided.

### DISCLOSURE OF INVENTION

### Technical Problem

Accordingly, the present invention has been made to solve at least the above-mentioned problems occurring in the prior art, and an abject of the present invention is to simply and easily provide a superior combinatorial Fab fragment library by consecutively transforming two vectors, which encode heavy and light chain fragments, in the form of circular DNA into host cells to alleviate problems with non-functional phage promiscuity and the existence of antibody clones having a loss of a part of antibody genes. Also, the present invention provides a method of constructing a plasmid vector (pLA-1 or pLT-2) for producing water-soluble light chain and heavy chain antibody fragments (VL+CL) and a phagemid vector (pHf1g3T-1 or pHf1g3A-2) having a (VH+CH1)-ΔpIII fusion protein expression and genotype-phenotype linkage function, transforming a host by using the vectors, and producing and selecting a water-soluble antibody and recombinant phage, which displays an antibody by using phage display technology, from the host. Additionally, the present invention provides a method of producing a combinatorial Fab fragment library (DVFAB-1L) by using DVS-II, and isolating Fab clones specific for four different antigens, which have an affinity of 10⁻⁶ to 10⁻⁷M, by biopanning against different antigens containing fluorescein-BSA. Further, the present invention provides a method of producing a huge combinatorial Fab library (DVFAB-131L) having a complexity of 1.5 10⁹ by a combination of 1 to 131 human kappa light and heavy chain repertoires, and identifying various fluorescein-BSA-specific heavy chains from the combinatorial Fab library.

### Technical Solution

In accordance with an aspect of the present invention, there is provided a method of producing pHf1g3T-1 phagemid, the method including the steps of:

(1) generating a DNA fragment by subjecting pBR322 plasmid to enzymatic hydrolysis with Pst I and EcoR I;

(2) generating a DNA fragment by subjecting pCMTG-SP112 phagemid to a PCR reaction with a primer set of Sequence No. 1 and Sequence No. 2 and subjecting a product of the PCR reaction to enzymatic hydrolysis with Pst I and Mun I;

(3) ligating the DNA fragments generated in steps (1) and (2);

(4) transforming electrocompetent TG1 cells by using the DNA fragments ligated in step (3); and

(5) culturing the TG1 cells transformed in step (4), and isolating and purifying phagemid from the cultured TG1 cells, and pHf1g3T-1 phagemid produced by the above method is also provided.

In accordance with another aspect of the present invention, there is provided a method of producing pLA-1 plasmid, the method including the steps of:

(1) generating a DNA fragment by subjecting pBAD/gIII plasmid to a PCR reaction with a primer set of Sequence No. 3 and Sequence No. 4 and subjecting a product of the PCR reaction to enzymatic hydrolysis with Cla I and Spe I;

(2) generating a DNA fragment by subjecting pCDFDuet-1 plasmid to a PCR reaction with a primer set of Sequence No. 5 and Sequence No. 6 and subjecting a product of the PCR reaction to enzymatic hydrolysis with Cla I and Spe I;

(3) ligating the DNA fragments generated in steps (1) and (2);

(4) transforming electrocompetent TG1 cells by using the DNA fragments ligated in step (3);

(5) culturing the TG1 cells transformed in step (4), and isolating and purifying plasmid from the cultured TG1 cells;

(6) generating a DNA fragment by subjecting the plasmid purified in step (5) to enzymatic hydrolysis with Nco I and Xho I;

(7) generating a DNA fragment by subjecting pCMTG-SP112 phagemid to a PCR reaction with a primer set of Sequence No. 7 and Sequence No. 8 and subjecting a product of the PCR reaction to enzymatic hydrolysis with Nco I and Xho I;

(8) ligating the DNA fragments generated in steps (6) and (7);

(9) transforming electrocompetent TG1 cells by using the DNA fragments ligated in step (8);

(10) culturing the TG1 cells transformed in step (9), and isolating and purifying plasmid from the cultured TG1 cells;

(11) generating a DNA fragment by subjecting the plasmid purified in step (10) to enzymatic hydrolysis with Sac I and Sac II;

(12) generating a DNA fragment by subjecting pCMTG-SP112 phagemid to a PCR reaction with a primer set of Sequence No. 9 and Sequence No. 10 and subjecting a product of the PCR reaction to enzymatic hydrolysis with Sac I and Sac II;

(13) ligating the DNA fragments generated in steps (11) and (12);

(14) transforming electrocompetent TG1 cells by using the DNA fragments ligated in step (13); and

(15) culturing the TG1 cells transformed in step (14), and isolating and purifying plasmid from the cultured TG1 cells, and pLA-1 plasmid produced by the above method is also provided.

In accordance with yet another aspect of the present invention, there is provided a method of producing pHf1g3A-2 phagemid, the method including the steps of:

(1) generating a DNA fragment by subjecting pLA-1 plasmid to enzymatic hydrolysis with Xho I and Sal I;

(2) generating a DNA fragment by subjecting pCMTG-SP112 phagemid to a PCR reaction with a primer set of Sequence No. 11 and Sequence No. 12 and subjecting a product of the PCR reaction to enzymatic hydrolysis with Xho I and Sal I;

(3) ligating the DNA fragments generated in steps (1) and (2);

(4) transforming electrocompetent TG1 cells by using the DNA fragments ligated in step (3); and

(5) culturing the TG1 cells transformed in step (4), and isolating and purifying phagemid from the cultured TG1 cells, and pHf1g3A-2 phagemid produced by the above method is also provided.

In accordance with still yet another aspect of the present invention, there is provided a method of producing pLT-2 plasmid, the method including the steps of:

(1) generating a DNA fragment by subjecting pBR322 plasmid to enzymatic hydrolysis with Pst I and EcoR I;

(2) generating a DNA fragment by subjecting pCMTG-SP112 phagemid to a PCR reaction with a primer set of Sequence No. 13 and Sequence No. 14 and subjecting a product of the PCR reaction to enzymatic hydrolysis with Pst I and Mun I;

(3) ligating the DNA fragments generated in steps (1) and (2);

(4) transforming electrocompetent TG1 cells by using the DNA fragments ligated in step (3); and

(5) culturing the TG1 cells transformed in step (4), and isolating and purifying phagemid from the cultured TG1 cells, and pLT-2 phagemid produced by the above method is also provided.

In accordance with still yet another aspect of the present invention, there is provided a dual vector system-I-A (DVS-I-A) including the steps of:

(1) transforming TG1 cells by using the pHf1g3T-1;

(2) transforming the TG1 cells, transformed in step (1), by using the pLA-1; and

(3) culturing the TG1 cells transformed in step (2).

In accordance with still yet another aspect of the present invention, there is provided a dual vector system-I-B (DVS-I-B) including the steps of:

(1) transforming TG1 cells by using the pLA-1;

(2) transforming the TG1 cells, transformed in step (1), by using the pHf1g3T-1; and

(3) culturing the TG1 cells transformed in step (2).

In accordance with still yet another aspect of the present invention, there is provided a dual vector system-II-A (DVS-II-A) including the steps of:

(1) transforming TG1 cells by using the pLT-2;

(2) transforming the TG1 cells, transformed in step (1), by using the pHf1g3A-2; and

(3) culturing the TG1 cells transformed in step (2).

In accordance with still yet another aspect of the present invention, there is provided a dual vector system-II-B (DVS-II-B) including the steps of:

(1) transforming TG1 cells by using the pHf1g3A-2;

(2) transforming the TG1 cells, transformed in step (1), by using the pLA-2; and

(3) culturing the TG1 cells transformed in step (2).

In accordance with still yet another aspect of the present invention, there is provided a method of expressing a human antibody Fab fragment gene by using the DVS-I-A, DVS-I-B, DVS-II-A, or DVS-II-B.

In accordance with still yet another aspect of the present invention, there is provided a method of producing a combinatorial Fab fragment library, DVFAB-1L or DVFAB-131L, by using the DVS-II-A or DVS-II-B to introduce pHf1g3A-2 phagemid DNA into TG1 cells containing pLT-2 plasmid with a single light chain or 1 to 131 light chains.

In accordance with still yet another aspect of the present invention, there is provided a method of selecting an antigen-specific human Fab fragment, the method including the steps of:

(a) panning phage with an antigen, the phage being obtained from a combinatorial Fab fragment library (DVFAB-1L) produced by the above method;

(b) obtaining TG1 cells containing pHf1g3A-2 phagemid DNA by infecting TG1 cells with the phage obtained in step (a);

(c) purifying pHf1g3A-2 phagemid DNA from the TG1 cells obtained in step (b);

(d) transforming TG1 cells containing pLT-2 plasmid, which encodes a single light chain, by using the phagemid DNA obtained in step (c); and

(e) superinfecting the TG1 cells transformed in step (d) with Ex 12 helper phage.

In accordance with still yet another aspect of the present invention, there is provided a method of selecting an antigen-specific htrnan Fab fragment, the method including the steps of:

(a) panning phage with an antigen, the phage being obtained from a combinatorial Fab fragment library (DVFAB-131L) produced by the above method;

(b) obtaining TG1 cells containing pHf1g3A-2 phagemid DNA by infecting TG1 cells with the phage obtained in step (a);

(c) purifying pHf1g3A-2 phagemid DNA from the TG1 cells obtained in step (b);

(d) transforming TG1 cells containing pLT-2 plasmid, which encodes 1 to 131 light chains, by using the phagemid DNA obtained in step (c); and

(e) superinfecting the TG1 cells transformed in step (d) with Ex 12 helper phage.

In accordance with still yet another aspect of the present invention, the antigen used in step (a) of the above methods is any one of fluorescein-BSA, GST (glutathione-S-transferase), biotin-BSA, and bSOD (bovine superoxide dismutase).

Important features of the above vectors according to the present invention are summarized below in Table 1.

TABLE 1
feature comparison between vectors

| | DVS-I | | DVS-II | |
|---|---|---|---|---|
| vector | pLA-1 | pHf1g3T-1 | pLT-2 | pHf1g3A-2 |
| plasmid and | plasmid | phagemid | plasmid | phagemid |
| phagemid | | | | |
| promoter | P_{BAD} | P_{lac} | P_{lac} | P_{BAD} |
| encoded antibody | light chain | Fd-ΔIII | light chain | Fd-ΔIII |
| fragments | | | | |
| signal sequence | gIII | ompA | ompA | gIII |
| derivative | arabinose | IPTG | IPTG | arabinose |
| replication origin | CDF or i | pBR or i | pBR or i | CDF or i |
| f1 origin | no | yes | no | yes |
| packaging into | no | yes | no | yes |
| phage progenies | | | | |
| antibiotic resistance | amp^{R} | tet^{R} | amp^{R} | tet^{R} |

In the specification, a dual vector system refers to a system for obtaining a target product by producing two vectors containing different foreign genes, and simultaneously or consecutively transforming one host with the two vectors. In particular, a system using host cells transformed with plasmid vector pLA-1 and phagemid vector pHf1g3T-1 will be referred to as “dual vector system (DVS)-I” and a system using host cells transformed with plasmid vector pLT-2-1 and phagemid vector pHf1g3A-2 will be referred to as “dual vector system (DVS)-II”.

### ADVANTAGEOUS EFFECTS

The present invention provides a phage display system including a dual vector system (DVS) by using pyruvate dehydrogenase complex-E2 (PDC-E2) specific SP112 Fab clone as a model. Also, the present invention can provide a combinatorial phage display Fab library by using the dual vector system.

Further, the dual vector system DVS-II of the present invention is practical in that it can more easily produce an antibody library with high diversity than the existing original phagemid vector without experimental problems with phage promiscuity and reduction in antibody concentration displayed on the surface of recombinant phage, and can be effectively used in new antibody-drug development because it has a possibility to develop a kit capable of selecting antibody genes humanized from various mouse antibody genes.

Further, the present invention provides a method of easily producing a combinatorial human antibody Fab fragment library by using the DVS-II system to bind a heavy chain repertoire to a very limited number of light chains, and isolating a single Fab fragment specifically binding to a target antigen. That is, the DVS-II system of the present invention more efficiently provides a combinatorial Fab fragment library with high diversity than when a normal single phagemid vector system is used, thereby reducing time and cost required for obtaining E. coli transformants in large quantities, and easily increasing the combinatorial Fab diversity of the DVFAB-131L 100 time or more as compared to the DVFAB-1L. It takes only a day to produce the DVFAB-131L with a Fab fragment diversity of 1.5×10⁹, which cannot be obtained using a normal single phagemid vector system. With regard to this, the combinatorial Fab diversity of the DVFAB-131L is easily increased 100 times or more as compared to the DVFAB-1L, and various fluorescein-BSA-specific heavy chains can be obtained from the DVFAB-131L.

### BRIEF DESCRIPTION OF THE DRAWINGS

The above and other objects, features and advantages of the present invention will become more apparent from the following detailed description taken in conjunction with the accompanying drawings, in which:

FIG. 1 is a schematic view of a vector produced in the present invention. FIG. 1A illustrates dual vector system-I (DVS-I) using a combination of pLA-1 plasmid and pHf1g3T-1 phagemid, and FIG. 1B illustrates dual vector system-II (DVS-II) using a combination of pLT-2 plasmid and pHf1g3A-2 phagemid.

FIG. 2 illustrates four different strategies for transforming electroccmpetent TG1 host cells in DVS-I (DVS-I-A or DVS-I-B) or DVS-II (DVS-II-A or DVS-II-B).

FIG. 3 illustrates a comparison between the numbers of E. coli colonies produced in dual vector systems according to different strategies. The colony formation unit (CFU) was measured from the number of E. coli colonies exhibiting phenotypes amp^{R} and tet^{R} after the cells are secondarily transformed according to FIG. 2. The order of introduction of vectors into non-transformed TG1 cells is as follows: (A) In DVS-I, vectors are introduced in order of DVS-I-A (pHf1g3T-1 →pLA-1) and DVS-I-B (pLA-1 →pHf1g3T-1); and (B) In DVS-II, vectors are introduced in order of DVS-II-A (pLT-2 →pHf1g3A-2) and DVS-II-B (pHf1g3A-2 →pLT-2). Data represents the average±standard deviation of three experiments.

FIG. 4 illustrates the antigen binding specificity of water-soluble SP112 molecules produced by TG1 cells carrying pCMTG-Sp112, DVS-I, and DVS-II. Other negative control antigens containing PDC-E2 and GST, IL-15, and BSA were coated on a microtiter plate. Supernatants were collected from media of the TG1 cells having pCMTG-Sp112, DVS-I, and DVS-II, and was applied to ELISA. A goat antihuman kappa light chain antibody, with which HRPO is conjugated, was used as a secondary antibody. Binding signals were visualized using TMB substrate, and were analyzed at OD₄₅₀ₙₘ. Data represents the average±standard deviation of three experiments.

FIG. 5 illustrates western blot analysis determining the expression of Fd-pIII and kappa light chains in TG1 host cells. SP112, DVS-I, and DVS-II were cultured in the presence of 0.1 mM of IPTG and 0.02% of arabinose. Whole cell lysates were obtained from precultured cells in order to obtain the same concentration, and were loaded into each well of 12% SDS-PAGE. Mouse anti-Myc tag mAb and AP-conjugated goat anti-mouse IgG was used as Fd-ΔpIII fusions (A), and AP-conjugated goat antihuman kappa light chain antibodies were used as kappa light chain fragments (B). They were visualized using NBT/BCIP substrate. Lane 1 represents TG1 cells having pCMTG-Sp112, lane 2 represents TG1 cells having DVS-I, and lane 3 represents TG1 cells having DVS-II.

FIG. 6 illustrates PFU measurement subsequent to obtaining phage from TG1 cells having different vector sets.

FIG. 7 illustrates phage ELISA representing antigen-specific binding of phage products obtained from TG1 cells having different vector sets.

FIG. 8 illustrates a schematic plan for affinity-guided selection of DVS-II.

FIG. 9 illustrates polyclonal phage ELISA determining PDC-E2-specific richness after consecutive panning rounds. TG1 cells having a positive control (pHf1g3A-2 and pLT-2) and TG1 cells having a negative control (pHf1g3A-2-BCKD and pLT-2) were used in ratios of 1:10⁴, 1:10⁶, and 1:10⁸, or were mixed with a negative control.

FIG. 10 is a schematic view of vector pBR322.

FIG. 11 is a schematic view of vector pCMTG. This is a vector with PDC-E2 antigen-specific VH and VL genes inserted into VH and VL gene positions.

FIG. 12 illustrates a method of producing DVFAB-1L and DVFAB-131L by using DVS-II.

FIG. 13 illustrates affinity-guided selection through DVFAB-1L and DVFAB-131L libraries.

FIG. 14 illustrates phage ELISA representing antigen-specific binding reactivity of phage obtained after each panning round using fluorescein-BSA (a), GST (B), biotin-BSA (C), or bSOD (D) as a target antigen. Recombinant phage of the same concentration (5×10⁷ PFU) was added into each well of a microtiter plate coated with fluorescein-BSA, glutathione-S-transferase, biotin-BSA, or bovine superoxide dismutase (bSOD). Bovine serum albumin (BSA) and L-glutamate dehydrogenase (L-Glu) were contained as a negative antigen. Phage particles binding to an antigen were detected by using anti-M13 Ab conjugated with HRPO as a secondary antibody. Binding was verified using TMB substrate, and was analyzed at OD₄₅₀ₙₘ. Data represents the average±standard deviation of three experiments.

FIG. 15 illustrates monoclonal ELISA for identifying E. coli clones producing target-specific Fab molecules. Water-soluble Fab molecules, which were produced by TGI cells obtained after the third panning round using fluorescein-BSA (A), GST (B), biotin-BSA (C), or bSOD (D) as a target antigen, was reacted with the same antigen as that used in panning. Goat antihuman kappa light chain Ab conjugated with HRPO was used as a secondary antibody. Binding was verified using TMB substrate, and was analyzed at OD₄₅₀ₙₘ.

FIG. 16 illustrates competitive inhibition ELISA for specifying the affinity of anti-fluorescein or anti-bSOD Fab. Culture supernatants containing water-soluble Fab were obtained from four E. coli clones expressing anti-fluorescein Fab (A) and six E. coli clones producing anti-bSOD Fab molecules (B), and were cultured with 10⁻⁵ to 10⁻¹²M of fluorescein (A) or bSOD (B) in advance. Subsequently, standard ELISA was carried out using an ELISA plate coated with fluorescein (A) or bSOD (B). The y-axis denotes the ratio of the ELISA signal (A450) in the absence of a solution-phage antigen to that in the presence of 10⁻⁵ to 0M of antigen. Data represents the average±standard deviation of three experiments.

FIG. 17 illustrates the derived amino acid sequences of anti-fluorescein-BSA or anti-bSOD Fab clones isolated from the DVFAB-1L library.

FIG. 18 illustrates the derived amino acid sequences of V_{H} genes of anti-fluorescein-BSA Fab clones isolated from the DVFAB-131L library.

FIG. 19 illustrates the derived amino acid sequences of V_{L} genes of anti-fluorescein-BSA Fab clones isolated from the DVFAB-131L library.

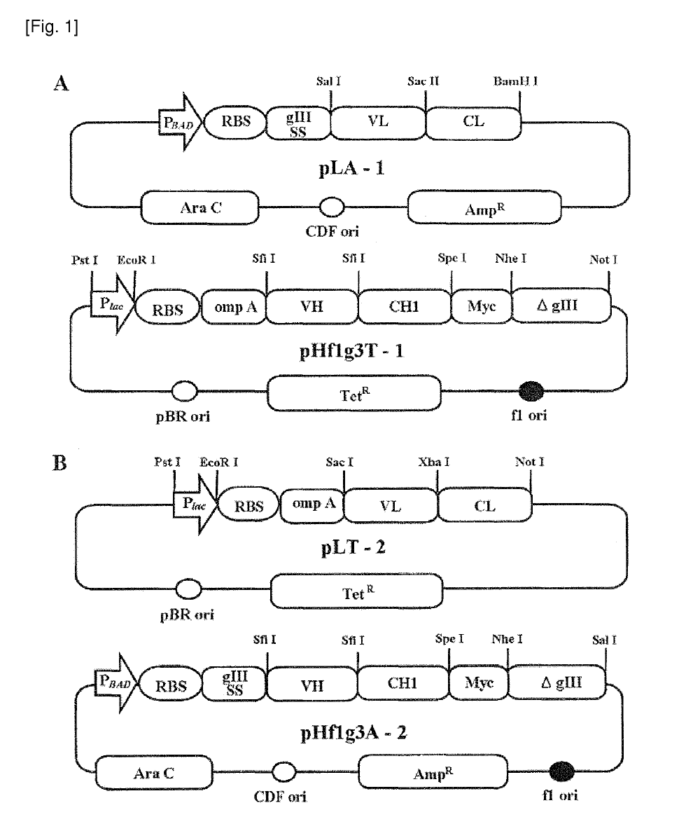

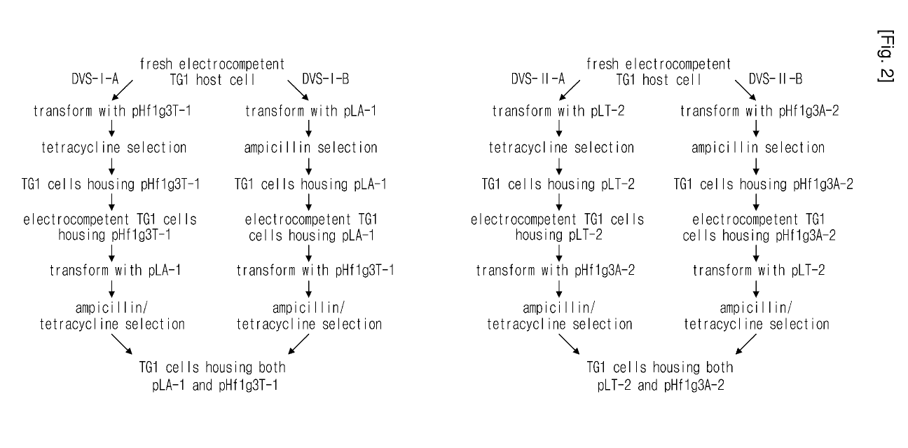

### MODE FOR THE INVENTION

Hereinafter, the present invention will be described in detail in conjunction with preferred embodiments, but the present invention is not limited thereto.

### Example 1

### Production of DVS-I and DVS-II

1.1 Bacterial Cell Line

An Escherichia coli cell line, TG1 (supE thi-1 Δlac-proAB)Δ(mcrB-hsdSM)₅ (rK-mK-)[F traD36 proAB lacIq lacZΔM15]) (Amersham Pharmacia Biotech, Sweden), was used as a bacterial host for cloning and recombinant phage production.

1.2 PCR Amplification and Oligonucleotide Synthesis

Ex-Tag polymerase (Takara, Japan) was used for all PCR amplifications, and all restriction enzymes were purchased from Takara, Japan. Also, all PCR primers used in the present invention were custom-synthesized by Bioneer, Korea.

1.3 Recombinant Vector Production

All DAN cloning experiments were carried out according to the standard method (Reference: J. Sambrook, E. F. Fritsch, T. Maniatis, Molecular Cloning, A laboratory Manual, 2^{nd} Ed., Cold Spring Harbor Laboratory Press, Cold Spring Harbor, N.Y., 199).

1.3.1 Dual Vector System-I (DVS-I) (Combination of pHf1g3T-1 Phagemid and pLA-1 Plasmid)

1.3.1.1 Production of pHf1g3T-1

pBR322 plasmid (provided by Dr. M. Eric Gershwin, University of California) was fragmented with Pst I and EcoR I, and a DNA fragment of 4 kb was obtained by performing electrophoresis using a 1% agarose gel and then by using the Wizard DNA cleanup kit (Promega, USA). Next, 30 units of CIP (calf intestinal phosphatase; Roche) were added to react with the DNA fragment in a 37° C. water bath for 1 hour, and then 1 μl of 0.5M EDTA was added to inactivate the reaction mixture at 65° C. for 1 hour. A DNA fragment including P_{lac}+Fd(V_{H}+C_{H1})+delta gIII+f1 on was amplified from pCMTS-SP112 (IG Therapy Co.) having Fab genes of SP112 corresponding to a PDC-E2-specific human monoclonal antibody by using a PCR method (sense primer: 5′-GGGCTGCAGACGCGGCCTTTTTACGGTGGTTCCT-3′ (Sequence No. 1), and anti-sense primer: 5′-GGGCAATTGCCGCGCACATTTCCCCGAAAAG-3′ (Sequence No. 2)) under the conditions of 35 cycles of 94° C. for 1 minute, 55° C. for 1 minute, and 72° C. for 1 minute, and under the condition of 72° C. for 10 minutes. In the PCR method, the Perkin Elmer 9700 machine (Perkin Elmer Inc.) was used as a PCR machine. The resultant PCR product was electrophoresed on a 1% agarose gel to isolate a DNA fragment (about 2.1 kb), and then was treated with restriction enzymes Pst I and Mun I. After the prepared pBR322 vector and PCR products were quantified, T4 DNA ligase (Takara) was added to react with them at 4° C., O/N (overnight), and electrocompetent TG1 cells were transformed with the reaction liquor by using the Gene-pulser II (Bio-rad, USA) under the conditions of 2.5 kV, 25, 0, and 200Ω. Subsequently, 1 ml LB medium was added to culture the transformed TG1 cells at 37° C. for 1 hour, and then the cultured cells were applied onto an LB agar plate containing 10 μg/ml tetracycline (LB/T plate) and were cultured at 37° C. overnight for antibiotic selection.

Phagemid was isolated and purified from the cultured cells to obtain pHf1g3T-1 phagemid.

1.3.1.2 Production of pLA-1 Plasmid Vector

A DNA fragment including AraC ORF+ara BAD promoter+Multicloning site AmpR ORF of pBAD/gIII (Invitrogen, USA, cat#: V45401) was amplified using a PCR method (sense primer: 5′-GGGATCGATTCAATTGTCT GATTCGTTACCAA-3′(Sequence No. 3), and anti-sense primer: 5′-GGGACTAGTTCAGTGGAA CGAAAACTCACG-3′ (Sequence No. 4)) under the conditions of 35 cycles of 94° C. for 1 minute, 55° C. for 1 minute, and 72° C. for 1 minute, and under the condition of 72° C. for 10 minutes. The resultant PCR product with a size of 2.9 kb was isolated using a 1% agarose gel, and was purified using the Wizard DNA cleanup kit. The obtained DNA fragment was treated with restriction enzymes Cla I and Spe I, and then was subjected to CIP treatment, as described above. Also, a gene fragment (about 850 bp) including CDF on was amplified from pCDFDuet-1 (Novagen, USA, cat#: 713443) by using a PCR method (sense primer: 5′-GGGATCGATATAGCTAGCTCACTCGGTCG-3′ (Sequence No. 5), and anti-sense primer: 5′-GGGACTAGTGCACTGAAATCTAGAGCGGAA-3′ (Sequence No. 6)) under the conditions of 35 cycles of 94° C. for 1 minute, 55° C. for 1 minute, and 72° C. for 1 minute, and under the condition of 72° C. for 10 minutes. The resultant PCR product was treated with restriction enzymes Cla I and Spe I and was mixed with the prepared pBAD/gIII vector fragment, and then T4 DNA ligase (Takara) was added to react with the mixture at 4° C., O/N (overnight). Electrocompetent TG1 cells were transformed with this reaction liquor by using the Gene-pulser II under the conditions of 2.5 kV, 25, 0, and 200Ω. Subsequently, 1 ml LB medium was added to culture the transformed TG1 cells at 37° C. for 1 hour, and then the cultured cells were applied onto an LB agar plate containing 50 μg/ml ampicillin (LB/A plate) and were cultured at 37° C. overnight for antibiotic selection. An E. coli strain was secured from the LB/A plate, a single colony was cultured in LB/A liquid medium, and then pBAD/gIII/CDF on recombinant plasmid was isolated and purified using the Wizard cleanup kit. This plasmid was treated with restriction enzymes Nco I and Xho I, and then was subjected to CIP treatment. Meanwhile, a hanan C_{L} kappa gene fragment with Sal I and Sac II cloning sites inserted into the 5′-terminal region was amplified from pCMTG-SP112 by using a PCR method (sense primer: 5′-GGGCCATGGGATTTAGGTGACACTATAGGATCTCGATCCCGCGAAAT-3′ (Sequence No. 7), and anti-sense primer:5′-GGGCTCGAGTTATCAACACTCTCCCCTGTTGCTC-3′ (Sequence No. 8)) under the conditions of 35 cycles of 94° C. for 1 minute, 55° C. for 1 minute, and 72° C. for 1 minute, and under the condition of 72° C. for 10 minutes. The resultant PCR product was treated with restriction enzymes Nco I and Xho I and was mixed with the above vector DNA at an appropriate concentration, and then T4 DNA ligase was added to react with the mixture at 4° C., O/N. Electrocompetent TG1 cells were transformed with this reaction liquor by using the Gene-pulser II under the conditions of 2.5 kV, 25 μF, and 200Ω. Subsequently, 1 ml LB medium was added to culture the transformed TG1 cells at 37° C. for 1 hour, and then the cultured cells were applied onto an LB/A agar plate and were cultured in a 37° C. incubator overnight for antibiotic selection. A single colony obtained in this way was cultured in LB/A liquid medium, and then plasmid with the human CL gene cloned thereinto was obtained using the Wizard plasmid cleanup kit. The obtained plasmid was treated with restriction enzymes Sac I and Sac II, 30 units of CIP were added to react with the plasmid in a 37° C. water bath for 1 hour, and then 1 μl of 0.5M EDTA was added to inactivate the reaction mixture at 65° C. for 1 hour. A human V_{L} gene fragment was amplified from pCMTG-SP112 by using a PCR method (V_{L} κaSal 5-GGGGTCGACA TGGACATCCAGATGAC-CCAGTCTCC-3′ (Sequence No. 9) and Jκac 5′-GGGCGGCGGATAC GTTTGATHTCCASYTTGGTCCC-3′ (Sequence No. 10)) (Degeneracy codons: H=A/C/T, S=G/C, Y═C/T) under the conditions of 35 cycles of 94° C. for 1 minute, 55° C. for 1 minute, and 72° C. for 1 minute, and under the condition of 72° C. for 10 minutes. The resultant PCR product was treated with restriction enzymes Sac I and Sac II and was mixed with the above vector DNA, and then T4 DNA ligase was added to react with the mixture at 4° C., O/N. Electrocompetent TG1 cells were transformed with this reaction liquor by using the Gene-pulser II under the conditions of 2.5 kV, 25 μF, and 200Ω. Subsequently, 1 ml LB medium was added to culture the transformed TG1 cells at 37° C. for 1 hour, and then the cultured cells were applied onto an LB/A agar plate and were cultured in a 37° C. incubator overnight for antibiotic selection. Plasmid was isolated and purified from the cultured cells to obtain pLA-1 plasmid.

1.3.2 Dual Vector System-II (DVS-II) (Combination of pHf1g3A-2 Phagemid and pLT-2 Plasmid)

1.3.2.1 Production of pHf1g3A-2 Phagemid Vector

This vector was produced from the pLA-1 vector produced in 1.3.1.2. The pLA-1 vector was treated with restriction enzymes Xho I and Sal Ito fragment a htrnan light chain antibody region, and then obtain a gene fragment of 4.3 kb. This gene was isolated using a 1% agarose gel, was purified using the Wizard DNA cleanup kit, and then was subjected to CIP treatment. A DNA fragment including Fd(VH+CH1)+ΔgIII+f1 on was amplified from pCMTG-SP112 by using a PCR method (sense primer: 5′-GGGCTGCAGACGCGGCCTTTTTACGGTGGTTCCT-3′ (Sequence No. 11), and anti-sense primer: 5′-GGGCAATTGCCGCGCACATTTCCCCGAAAAG-3′ (Sequence No. 12)) under the conditions of 35 cycles of 94° C. for 1 minute, 55° C. for 1 minute, and 72° C. for 1 minute, and under the condition of 72° C. for 10 minutes. The resultant PCR product was treated with restriction enzymes Xho I and Sal I and was mixed with the above vector DNA in a molar ratio of 1:2, and then T4 DNA ligase was added to react with the mixture at 4° C., O/N. Electrocompetent TG1 cells were transformed with this reaction liquor by using the Gene-pulser II under the conditions of 2.5 kV, 25 μF, and 200Ω. Subsequently, 1 ml LB medium was added to culture the transformed TG1 cells at 37° C. for 1 hour, and then antibiotic selection was carried out using an LB/A agar plate.

The antibiotic-selected cells were cultured, and phagemid was isolated and purified from the cultured cells to obtain pHf1g3A-2 phagemid.

Meanwhile, pHf1g3A-2-BCKD for use as a negative control in biopanning experiments was separately produced by replacing the Fd(VH+CH1) genes of PDC-E2-specific SP112 existing in pHf1g3A-2 with the Fd(VH+CH1) genes (IG Therapy Co.) of a BCKD-E2 (branched-chain alpha-keto acid dehydrogenase complex-E2)-specific antibody.

1.3.2.2 Production of pLT-2 Plasmid Vector

This vector was produced using pBR322. Plasmid vector pBR322 (provided by Dr. M. Eric Gershwin, University of California) was treated with restriction enzymes Pst I and EcoR Ito isolate a gene fragment of 3.6 kb from a 1% agarose gel, purify the isolated gene fragment by using the Wizard DNA cleanup kit, and then treat the purified gene fragment with CIP. A DNA fragment including P_{lac}+SP112 light chain genes was amplified from pCMTG-SP112 by using a PCR method (sense primer: 5′-GGGATCGATTCAATTGTCTGATTCGTT ACCAA-3′ (Sequence No. 13), and anti-sense primer: 5′-GGGACTAGTTCAGTGGAACGAAAACTC ACG-3′ (Sequence No. 14)) under the conditions of 35 cycles of 94° C. for 1 minute, 55° C. for 1 minute, and 72° C. for 1 minute, and under the condition of 72° C. for 10 minutes. The resultant PCR product was treated with restriction enzymes Pst I and Mun I and was mixed with the prepared pBR322 vector in a molar ratio of 1:2, and then T4 DNA ligase was added to react with the mixture at 4° C., O/N. Electrocompetent TG1 cells were transformed with this reaction liquor by using the Gene-pulser II under the conditions of 2.5 kV, 25 μF, and 200Ω. Subsequently, 1 ml LB medium was added to culture the transformed TG1 cells at 37° C. for 1 hour, and then antibiotic selection was carried out using an LB/T agar plate.

The antibiotic-selected cells were cultured, and plasmid was isolated and purified from the cultured cells to obtain pLT-2 plasmid.

### Example 2

### E. coli Transformation

2.1 Transformation Experiment for Dual Vector System-I (DVS-I)

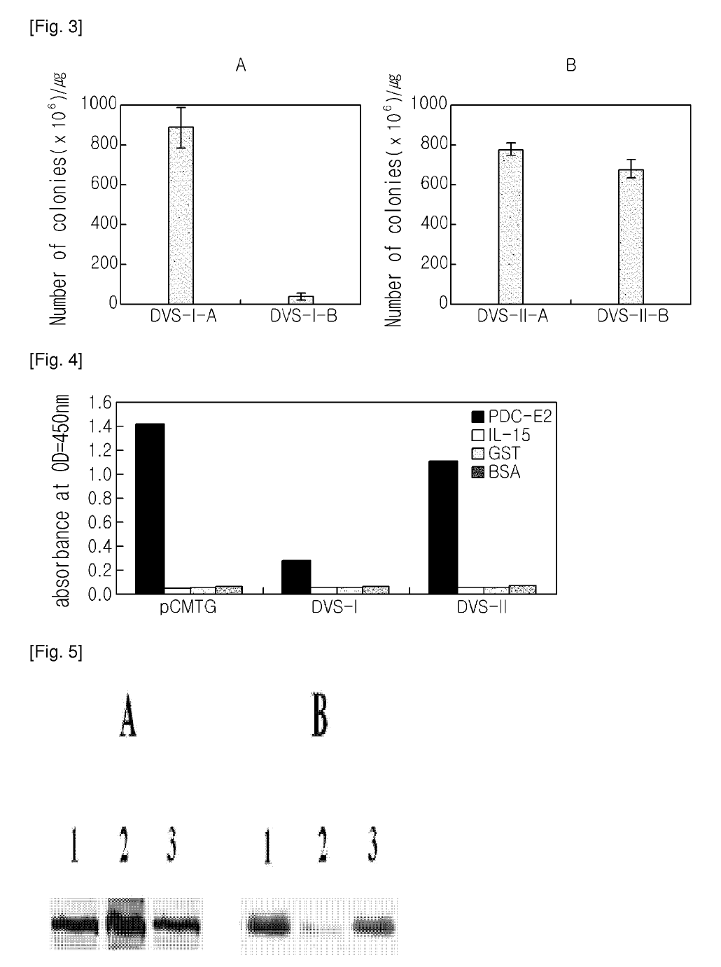

Fresh TG1 E. coli was cultured in LB medium, and then was centrifuged at 4000 g for 15 minutes by means of the J2-MC centrifuge (Beckman). The supernatant was removed, and TG1 cells were washed using sterile distilled water containing 10% glycerol (Duchefa). Such a procedure was repeated three times to produce an electro-competent cell line, and then its TG1 cells were transformed with 100 ng of vector pLA-1 or pHf1g3T-1 by using the Gene-pulser II under the conditions of 2.5 kV, 2 μF, and 200Ω. Subsequently, the TG1 cells were applied onto LB/A and LB/T plates respectively, and were cultured at 37° C. Cells containing pLA-1 and cells containing pHf1g3T-1 were selected from the generated E. coli colonies, and then were grown up to OD₆₀₀=0.5 in 2% glucose (Duchfa)-containing LB/A (LB/AG) or LB/T (LB-TG) medium. The respective cultured cells were centrifuged at 4000×g for 15 minutes by means of the J2-MC centrifuge (Beckman), each supernatant was removed, and then each remainder was washed using sterile distilled water containing 10% glycerol. Such a procedure was repeated three times to produce electrocompetent TG1 cells containing pLA-1 or pHf1g3T-1. Subsequently, the electrocompetent TG1 cell line containing pLA-1 was transformed by electroporation with 100 ng of pHf1g3T-1, and the electrocompetent TG1 cell line containing pHf1g3T-1 was transformed by electroporation with 100 ng of pLA-1. The completely transformed cells were cultured on an LB/AT plate containing both ampicillin and tetracycline at 37° C., O/N for antibiotic selection. The numbers of the respective colonies generated after the culture were measured to determine CFUs (colony forming units), which are illustrated in FIG. 3A.

As illustrated in FIG. 3A, about 9×10⁸CFU/μg DNA was obtained when the TG1 host cells containing pHf1g3T-1 phagemid were transformed using the pLA-1 plasmid (DVS-I-A), but the transformation efficiency of the TG1 host cells decreased about 45 times when the order of introduction of the vectors into the host cells was transposed (DVS-I-B). From this it can be seen that there is a significant difference in trans-formation efficiency according to the order of introduction of vectors into host cells.

2.2 Transformation Experiment for Dual Vector System-II (DVS-II)

A TG1 cell line was transformed with 100 ng of vector pLT-2 or pHf1g3A-2 by using the Gene-pulser II under the conditions of 2.5 kV, 25 μF, and 200Ω. After the transformation, cells containing pLT-2 or pHf1g3A-2 were selected from E. coli colonies generated by applying the transformed TG1 cells onto an LB/A or LB/T plate and culturing them at 37° C., and then the selected cells were grown up to OD₆₀₀=0.5 in 2% glucose-containing LB/TG or LB/AG medium. The cultured cells were centrifuged at 4000 g for 15 minutes by means of the J2-MC centrifuge, the supernatant was removed, and then the remainder was washed using sterile distilled water containing 10% glycerol. Such a procedure was repeated three times to produce an electro-competent TG1 cell line into which pLT-2 or pHf1g3A-2 was inserted. Subsequently, the electrocompetent TG1 cell line containing pLT-2 was transformed by electroporation with 100 ng of pHf1g3A-2, and the electrocompetent TG1 cell line containing pHf1g3A-2 was transformed by electroporation with 100 ng of pLT-2. The completely transformed cells were cultured on an LB/AT plate at 37° C., O/N for antibiotic selection. The numbers of the respective colonies generated after culturing were measured to determine CFUs (colony forming units), which are illustrated in FIG. 3B.

As illustrated in FIG. 3B, the numbers of TG1 cells exhibiting phenotypes amp^{R} and tet^{R} were 7.8×10⁸CFU/μg DNA and 6.7×10⁸CFU/μg in DVS-II-A and DVS-II-B respectively, that is, were almost similar in both the systems. From this it can be seen that the transformation efficiency of host cells is hardly affected by the order of introduction of vectors pHf1g3A-2 and pLT-2 in DVS-II, and thus DVS-II has higher vector stability than that in DVS-I.

### Example 3

### ELISA for Water-Soluble Fab Molecules

To prepare water-soluble Fab molecules, TGI cells, into which pCMTG-SP112, DVS-I, or DVS-II was inserted, were cultured under the following conditions: 10 ml of LB/AG medium was used for pCMTG-SP112, 10 ml of LB/ATG medium was used for DVS-I and DVS-II, and the TG1 cells were cultured up to OD₆₀₀=0.5. Each culture was centrifuged at 3300×g for 10 minutes, and then each supernatant was removed. Subsequently, pCMTG-SP112 was resuspended using 0.1 mM IPTG (isopropyl-β-D-1-thiogalactopyranisid)-added LB/A medium (LB/AI), DVS-I and DVS-II were resuspended using LB/A medium containing

0.02% arabinose and 0.1 mM IPTG (LB/ATIA), and then the suspension was cultured at 27° C. for 15 hours. Each culture was centrifuged to obtain the supernatant containing water-soluble Fab. Each of 10 μg/ml PDC-E2, glutathione-S-transferase (GST), human interleukin-15 (IL-15), and bovine serum albumin (BSA) was diluted with coating buffer (0.1M NaHCO₃, pH 9.6), was added as an antigen to the Maxi-sorp immunoplate (Nunc. Denmark) in an amount of 50 μl per well, and then was adsorbed at 4° C., O/N. The plate was washed with 0.1% Tween-containing phosphate-buffered saline (PBS-Tween) three times, and then 200 μl of blocking buffer (PBS containing 3% skimmed milk) was added to react with each antigen at 37° C. for 1 hour. After the plate was washed with PBS-Tween three times again, 50 μl of the obtained water-soluble Fab supernatant was added into each well to react with the antigen at 37° C. for 1 hour. After the plate was washed with PBS-Tween three times, goat antihuman kappa light chain antibody-HRPO-conjugated pAb (Sigma) diluted to 1:5000 with blocking buffer was added to the plate, and then whether or not each water-soluble Fab fragment has specific reactivity to PDC-E2 was verified. A binding reaction was confirmed using 3.3′5.5′tetramethyl bezidine (TMB) substrate, absorbance at 450 nm was measured using an ELISA reader (Biorad). The results are illustrated in FIG. 4.

As can seen from FIG. 4, DVS-I produced SP112 Fab molecules at a level that was about ⅕ or less as compared to pCMTG-SP112, but DVS-II showed no difference in the amount of SP112 Fab fragment production as compared to pCMTG-SP112 and produced an Fab fragment with antigen-binding activity at a level that is about four times as large as DVS-I. Also, Fab molecules existing in the above three TG1 cell cultures did not bind to negative control antigens (IL-15, GST, and BSA).

Meanwhile, phage ELISA was carried out in the same manner as described above while 50 μl of phage supernatant (5×10⁷ PFU/well) was added to react with the antigen at 37° C. for 1 hour. After the plate was washed with PBS-Tween, goat anti-M13 HRPO-conjugated pAb (Sigma) diluted to 1:5000 with blocking buffer was added to the plate, and then whether or not phage has specific reactivity to PDC-E2 was verified. The results are illustrated in FIG. 7.

As can seen from FIG. 7, all recombinant phage produced by pCMTG-SP112, DVS-I, and DVS-II exhibited specific binding activity to PDC-E2, and did not react with the negative control antigens (IL-15, GST, and BSA).

### Example 4

### Comparison of Amount of Fab Fragment Expression through Western Blot Assay

TG1 cells containing recombinant vectors (pCMTG-SP112, DVS-I, and DVS-II) were cultured in medium containing IPTG and arabinose, as mentioned in Example 3, and then the cell sediment was obtained by centrifugation. The obtained sediment was resuspended with SDS-sample buffer in a ratio of 1:1 and was heated in boiling water for 5 minute, and then the 12% SDS-PAGE experiment was carried out. Thereafter, proteins existing in SDS-PAGE were transferred to a nitrocellulose membrane (Amersham Pharmacia biotech) by using the Ready gel precast gel system (Biorad) at 65V for 90 minutes. The membrane with the proteins transferred thereto reacted with blocking buffer at room temperature for 1 hour, was washed with PBS-Tween three times for each 5 minutes, and then mouse anti-myc mAb (IG Therapy Co.) diluted to 1:3000 with blocking buffer reacted with the membrane at room temperature for 1 hour in order to detect fused Fd-ΔpIII. After the membrane was washed with PBS-Tween three times for each 5 minutes again, goat anti-mouse IgG AP-conjugated pAb (Sigma) diluted to 1:5000 with blocking buffer reacted with the membrane for 1 hour. Meanwhile, goat antihuman kappa light chain AP-conjugated pAb (Sigma) was used to detect human light chain fragments. Nitro blue tetraxthum chloride (NBT)/5-brow-4-chloro-3-indolliphosphate (BCIP) substrate (Sigma) was used as substrate, and signals appearing on the membrane were analyzed using a densitometer (Biorad), the results of which are illustrated in FIG. 5.

As seen from FIG. 5, within the TG1 host cells, DVS-I produced Fd-ΔpIII molecules at a level that is about three or four times as large as pCMTG-SP112 and DVS-II, but expressed human light chain fragments at a level that is about ⅙ to 1/10 as compared to pCMTG-SP112 and DVS-II. Thus, since Fab having antigen-binding capability is optimally produced by a combination of Fd and light chain fragments having the same number of molecules, it is inferred that low production of Fab molecules with antigen binding activity, exhibited by DVS-I, is caused by unbalanced expression of antibody fragments constituting Fab molecules.

### Example 5

### Amplification of Recombinant Phage

Using a method that was modified by making reference to amplification of recombinant phage, reported in the prior art (References: McCafferty, J., 1996, Phage display: factors affecting panning efficiency. In: Kay, B. K., Winter, J., McCafferty, J. (Eds.), Phage Display of Peptides and Proteins, a Laboratory Manual, Academic Press, San Diego, p. 261; Baek, H., Suk, K. H., Kim, Y. H., Cha, S., 2002, An improved helper phage system for efficient isolation of specific antibody molecules in phage display, Nucleic Acids Res. 30(5), e18), recombinant phage was obtained as follows: In brief, TG1 cells containing each recombinant vector (pCMTG-Sp112, DVS-I, or DVS-II) were first cultured. pCMTG-Sp112 was grown in 10 ml of LB/AG medium, and DVS-I and DVS-II were grown up to OD₆₀₀=about 0.5 in 10ml of LB/ATG medium. Each culture was centrifuged at 3300×g for 10 minutes, and then was resuspended with 10ml of LB/G medium. M13K07 or Ex-12 helper phage was added to the suspension at 20 MOI (multiplicity of infection), and then the suspension was cultured at 37° C. for 1 hour. A mixed liquor of the cell line and the helper phage was centrifuged at 3300×g for 10 minutes again, and then the supernatant was removed to obtain cells. Subsequently, pCMTG-SP112 was resuspended with 100ml of LB/AK (containing 100 μg of ampicillin and 50 μg of kanamycin), DVS-I and DVS-II were resuspended with 100ml of LB/ATKA (containing 100 μg of ampicillin, 10 μg of tetracycline, 50 μg of kanamycin, and 0.001% arabinose), and then the suspension was cultured at 27° C. for 15 hours. The culture was centrifuged at 3300×g for 20 minutes to then obtain the supernatant containing recombinant phage. Phage particles were sedimented using a PEG/NaCl solution, and then were resuspended with 1 ml of sterile PBS to obtain enriched phage.

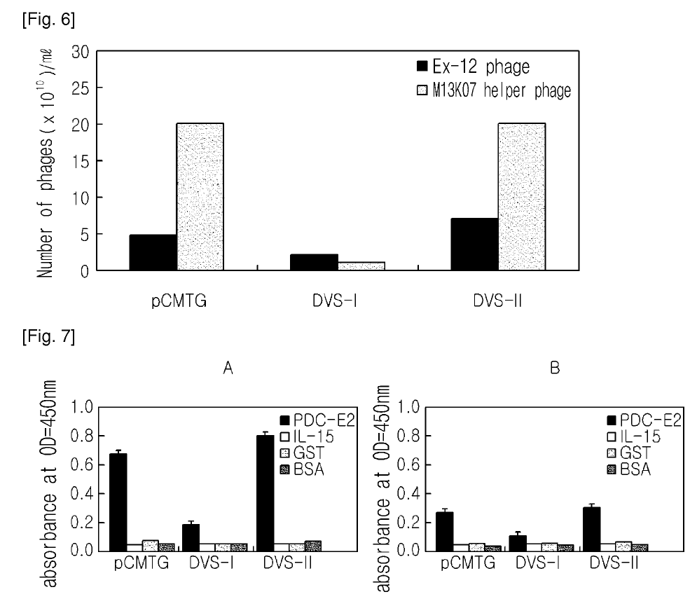

Phage titer was measured by the following PFU (plaque forming unit) assay: TG1 cells were cultured up to OD₆₀₀=0.8 in LB medium, 1 μl of the obtained phage concentrate was added to and mixed with 100 μl of the culture, and then the mixture was 100-fold diluted step by step to 10⁻², 10⁻⁴, 10⁻⁶, and 10⁻⁸ with the TG1 cell medium. After the mixture reacted at 37° C. for 30 minutes, the reaction liquor was mixed with 4ml of top agar, and the mixture was flatly poured onto an LB plate. The plate left at room temperature for 10 minute was cultured at 37° C. for 15 hours, and the titer of the obtained phage was calculated by measuring plaque generated on the plate, the results of which are illustrated in FIG. 6.

As seen from FIG. 6, when Ex-12 helper phage was used for phage rescue, DVS-II exhibited the highest phage titer (about 7×10¹⁰ PFU/ml), and pCMTG-SP112 and DVS-I produced phage at levels of about 5×10¹⁰ PFU/ml and 2×10¹⁰ PFU/ml respectively. That is, DVS-II exhibits the best recombinant phage productivity. In the case of M13K07 helper phage, while pCMTG-SP112 and DVS-II exhibited a similar phage titer of about 2×10¹¹ PFU/ml, DVS-I exhibited a phage titer of about 10¹⁰ PFU/ml. Thus, as compared to pCMTG-SP112 and DVS-II, DVS-I exhibited recombinant phage productivity lowered 2 to 3 times for Ex-12 helper phage and lowered 20 times for M13K07 helper phage.

Also, recombinant phage production for biopanning was also carried out in a manner as described above, except that a strain obtained by diluting TG1 cells, into which DVS-II was inserted, in a ratio of 1:10⁴, 1:10⁶, or 1:10⁸ with TG1 cells containing DVS-II-BCKD (pLT-2 and pHf1g3A-2-BCKD) was used, and Ex-12 helper phage was used as helper phage.

### Example 6

### Biopanning

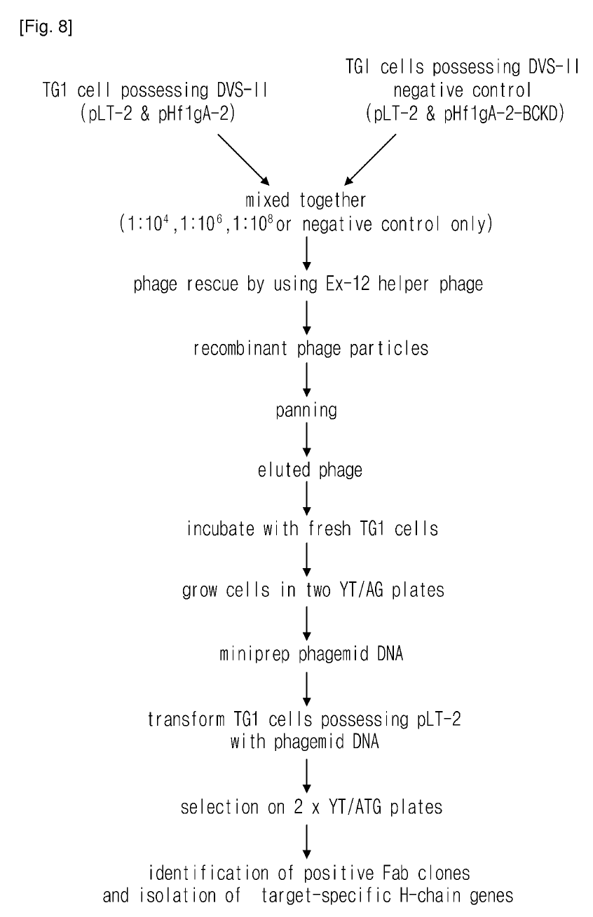

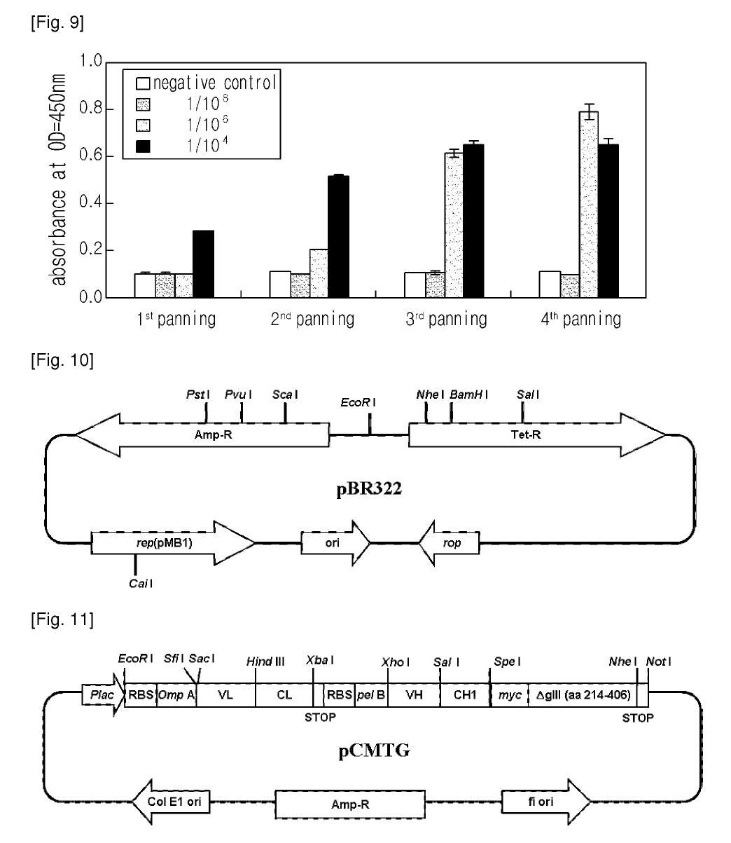

Selection of recombinant phage binding to PDC-E2 was carried out by a panning method as schematically illustrated in FIG. 8 (Reference: Baek, H., Suk, K. H., Kim, Y. H., Cha, S., 2002, An improved helper phage system for efficient isolation of specific antibody molecules in phage display, Nucleic Acids Res. 30(5), e18). First of all, a 10 μg/ml PDC-E2 antigen reacted with the Maxi-sorp immunoplate by using coating buffer at 4° C., O/N. Subsequently, the plate was washed with PBS-Tween three times, and 200 μl of blocking buffer was added to react with the antigen at 37° C. for 1 hour. Recombinant phage was obtained from a sample in which TG1 cell lines having DVS-II (i.e. positive control) and DVS-II-BCKD (i.e. negative control) inserted therein respectively were mixed and cultured in a ratio of 1:10⁴, 1:10⁶, or 1:10⁸, the recombinant phage was added into 24 microwells at a total concentration of 1.2×10⁹ (5×10⁷/well), and then the recombinant phage reacted with the antigen at 37° C. for 2 hours. After the plate was washed with PBS-Tween ten times, the phage was eluted from the plate by adding 500 of elution buffer (0.1M glycine-HCl, pH 2.5) into each microwell to react with the phage for 10 minutes. Fresh TG1 cells were infected with the obtained phage, and then the infected cells were applied onto an LB/T plate and were culture at 27° C. overnight. E. coli colonies grown on the plate were obtained using a sterilized glass rod, and pHf1g3A-2 phagemid DNA was purified from the colonies by using the Wizard plasmid cleanup kit. 100 ng of this phagemid DNA was introduced into TG1 cells, into which pLT-2 was already inserted, by electroporation, the TG1 cells were applied onto an LB/AT plate to select TG1 cell lines, and then the recombinant phage was amplified again from the selected cell lines by using Ex-12 helper phage. The recombinant phage amplified in this way was used for the panning again, and such an experiment was repeated four times in total. For recombinant phage obtained each step and E. coli clones, binding reactivity with PDC-E2 was measured through ELISA, the results of which are illustrated in FIG. 9.

As seen from FIG. 9, PDC-E2-specific selection was performed from the first panning under the condition that the negative control, that is, DVS-II-BCKD, was 10⁴ times as many as DVS-II, and PDC-E2-specific selection was performed from the second panning under the condition that the negative control, that is, DVS-II-BCKD, was 10⁶ times as many as DVS-II. However, under the condition that the negative control, that is, DVS-II-BCKD, was 10⁸ times as many as DVS-II, PDC-E2-specific selection of the recombinant phage was not performed, even when up to the fourth panning was carried out. In order to confirm PDC-E2-specific selection of the recombinant phage, which appeared in the phage ELISA, at the clone level, phagemid genome was isolated from recombinant phage obtained after each panning round and was inserted into TG1 cells containing pLT-2 to obtain E. coli colonies. 24 colonies among the obtained E. coli colonies were randomly cultured, and then the culture was subjected to ELISA to examine if each E. coli clone produces a PDC-E2-specific Fab fragment, the results of which are given below in Table 2.

TABLE 2
frequency of E. coli clones secreting anti-PDC-E2 Fab molecules
after each panning round

| dosing | yield rate of anti-PCD-E2 clones^{a} | | | |
|---|---|---|---|---|
| rate (positive/negative) | 1^{st} round | 2^{nd} round | 3^{rd} round | 4^{th} round |
| 1:10⁴ | 4/24 | 24/24 | 24/24 | 24/24 |
| 1:10⁶ | 0/24 | 4/24 | 21/24 | 24/24 |
| 1:10⁸ | 0/24 | 0/24 | 0/24 | 0/24 |
| negative control | 0/24 | 0/24 | 0/24 | 0/24 |

^{a}24 clones were randomly extracted for antigen binding ELISA. In this table, data represents the ratio of (no. of positive clones/24 clones).

As seen from Table 2, all 24 clones obtained after the second panning produced a PDC-E2-specific Fab fragment under the condition that the negative control, that is, DVS-II-BCKD, was 10⁴ times as many as DVS-II, and all clones obtained after the fourth panning produced a PDC-E2-specific Fab fragment under the condition that the negative control, that is, DVS-II-BCKD, was 10⁶ times as many as DVS-II. This is consistent with the results of the phage ELISA in FIG. 9, and proves that selection of antigen-specific recombinant phage is advanced about 100 times per panning round.

In summary, DVS-II was confirmed to have stable transformation efficiency of host cells regardless of the order of introduction of vectors into the host cells, as compared to DVS-I. Also, in the case of using DVS-II, the amount of expression of water-soluble Fab molecules with antigen binding reactivity, the titer of recombinant phage, and the amount of Fab-ApIII displayed on the surfaces of phage progenies were similar to those of the existing conventional phage display system using a single phagemid vector, and recombinant phage displaying target-specific Fab-ΔpIII molecules could be successfully selected using panning, so that antigen-specific Fd gene could be isolated from pHf1g3A-2 phagemid.

### Example 7

### Generation of Combinatorial Human Antibody Fab Fragment Library

7.1 Production of Human Heavy Chain Sub-library

Natural human Fd (V_{H}+C_{H1}) genes obtained in advance from peripheral blood lymphocytes of 40 applicants was cloned into vector pCMTGAK (IG Therapy, South Korea) in which kanamycin resistant gene is located downstream of Fd gene. Ligated vector pCMTGAK was introduced into XL-1 Blue E. coli cells (Stratagene, USA) by electroporation, and 2 millions of E. coli transformants exhibiting kanamycin resistant phenotype were selected. Fd gene was isolated from the E. coli transformants, was sub-cloned into vector pCMTG (IG Therapy), and was used as a PCR template. Natural and semi-synthetic V_{H} gene repertoires were obtained by PCR amplification over 20 cycles of 94° C. for 1 minute, 56° C. for 1 minute, and 72° C. for 1 minute. HuVH sense and HuJH anti-sense primers were used to produce a natural heavy chain repertoire (HuVH sense: 5′-GCAACTGCGGCCCAGCCGGCC AT GGCCSAGGT-GCAGCTGKTGCAGTCTGG-3′, and HuJH anti-sense: 5′-GGGGGCCAATGTGGCC GAT GAGGAGACGGTGACCAKGGTBCCTTGGCCCCA-3′) (non-complementary Sfi I restriction enzyme sites are written in italics, and degeneracy is designated by S=G or C; K=G or T; and B=G, T, or C). To obtain a semi-synthetic heavy chain repertoire, HuVH sense and HuJH-syn anti-sense primers (HuJH-syn anti-sense: 5′-TGAGGAGACGGTGACCAKGGTBCCTTGGCCCCAAWMRDY (SNN)₄₋₈ GCGTGCACAG TACACGGCCGTGTC-3′, where degeneracy is designated by W=A or T; M=A or C; R=G or A; D=G, A, or T; Y═C or T; N=A, G, T, or C) and 157 natural V_{H} frameworks that are translated well in E. coli (IG Therapy Co.) were used in the first PCR round, and then the PCR product of 350 bp was purified using the Wizard DNA cleanup system (Promega, USA). The second PCR round was carried out using HuVH sense and HuJH anti-sense primers under the same condition as described above. Natural or semi-synthetic human V_{H} gene produced in this way and pHf1g3A-3phagemid were subjected to enzymatic hydrolysis with restriction enzyme Sfi I and were ligated together using T4 DNA ligase (Takara) to produce a heavy chain sub-library. The ligated DNA product was extracted with phenol/chloroform, was sedimented with ethanol, and then was electroporated into E. coli ElectroTen Blue cells (Stratagene, USA) by using the Gene Pulser II (Biorad, USA) set to 2.5 kV, 25 μF, and 200 W. The transformed cells were applied onto a 2×YT plate containing 50 μg/ml ampicillin and 10 μg/ml carbenicillin (2×YT/ACG), and were cultured at 27° C. overnight. Colonies generated on the plate were obtained together with 2×YT medium added to the plate. Subsequently, pHf1g3A-2 phagemid DNA was purified from the cells by using the Wizard plus SV minipreps kit (Promega).

7.2 Natural Human Light Chain Isolation and Cloning

The whole RNA was produced from human peripheral blood red cells by using Trizol (Invitrogen, USA), and first strand cDNA was synthesized using the olig-dT primer and the First strand cDNA synthesis kit (Roche, Germany). Subsequently, a V_{L} gene fragment was obtained by PCR amplification using HuVLk and HuJk primers (HuVLk1 sense: 5′-GGGGAGCTCGACATCCAGWTGACCCAGTCTCC-3′, HuVLk2 sense: 5′-GGGGAGCTCGAAATTGTGTTGACRCAGTCTCC-3′, HuVLk3 sense: 5′-GGGGAGCTCGATATTGT GATGACYCAGTCTCC-3′, HuVLk4 sense: 5′-GGG GAGCTCGTGTTGACGCAGTCTCCAGGCAC-3′, and HuJk anti-sense: 5′-CACAGT TCTAGAACGTTTRATHTCCASYYKKGTCCC-3′, where degeneracy is designated by H=A, C, or T, and Sac I and Xba I restriction enzyme sites are written in italics) over 20 cycles of 94° C. for 1 minute, 56° C. for 1 minute, and 72° C. for 1 minute. The PCR product of 350 bp was purified using the Wizard PCR cleanup kit, and was treated with Sac I and Xba I. Vector pLT-2 was also treated with the same restriction enzymes, and then was ligated to the V_{L} gene inserted therein. The produced ligation reaction product was introduced into E. coli TG1 cells (Stratagene, USA) by electroporation, and the generated transgenic cells were applied onto a 2×YT plate containing 10 μg/ml tetracylin (2×YT/T) and were cultured at 27° C. overnight. 400 or more colonies were added in 200 μl of 2×YT/T medium with 0.1 mM IPTG (isopropyl-(3-D-1-thiogalactopyranisid) added thereto, and 131 E. coli clones producing water-soluble kappa light chains were selected by ELISA using HRPO (horse radish peroxidase) (Sigma-Aldrich, USA)-conjugated goat antihuman kappa light chain pAb. In order to produce a combinatorial Fab library, these cells were grown up to OD₆₀₀=about 0.4 in 2×YT/T medium and were thoroughly washed with 10% glycerol-containing ddH₂O to be made electrocanpetent.

7.3 Production of Combinatorial Fab Fragment Library DVFAB-1L and DVFAB-131L

7.3.1 Production Process

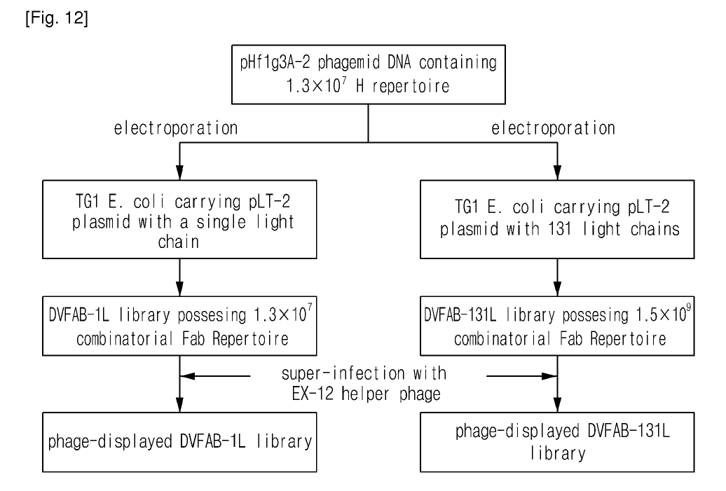

Electrocompetent TG1 cells including pLT-2 that has single or 131 independent natural human light chains were transformed with 2 or 20 μg of human heavy chain repertoire-containing pHf1g3A-2 phagemid (containing human heavy chain gene with a diversity of 1.3×10⁷) to produce DVFAB-1L or DVFAB-131L library containing a human heavy chain repertoire (FIG. 12). TG1 cells (Stratagene, cat#: 200123) were prepared and used as host cells for electrophoresis. Selection was performed by culturing them at 37° C. for 8 hours in 2×YT/ACTG medium containing 2% glucose, 50 μg/ml ampicillin, 10 μg/ml carbenicillin, and 10 μg/ml tetracylin. Subsequently, the TG1 cells were moved to 500ml of fresh 2×YT medium containing 100ml of medium (2×YT/ACTG), and were cultured up to OD₆₀₀=about 0.5 at 37° C. Next, the bacterial cell culture was centrifuged at 3300×g for 10 minutes, and then the produced cell pellets were resuspended up to 20 MOI (multiplicity of infection) with 500 ml of fresh 2×YT medium (2×YT/G) containing 2% glucose and Ex-12 helper phage (IG Therapy) and were cultured 37° C. for 1 hour for Phage rescue (References: Baek, H. J., Suk, K. H., Kim, Y. H. and Cha, S. H., (2002), An improved helper phage system for efficient isolation of specific antibody molecules in phage display, Nucleic Acids Res., 30, e18; Oh, M. Y., Joo, H. Y., Hur, B. U., Jeong, Y. H. and Cha, S. H, (2007), Enhancing phage display of antibody fragments using gIII-amber suppression, Gene, 386, 81-89). Subsequently, the culture was centrifuged at 3300×g for 10 minutes, and then the produced cell pellets were resuspended with 5 L of fresh 2×YT/AT medium (2×YT/ATKT) supplemented with 70 μg/ml kanamycin and 0.001% arabinose (w/v). After the suspension was cultured 27° C. overnight, recombinant phage particles were obtained by centrifuging the culture at 3300×g for 20 minutes. The phage supernatant was sterilized using a 0.45 μm filter, and 40ml of Aliquart was prepared for long-term storage at −80° C. Final phage in the 40ml of storage solution was sedimented with PEG/NaCl solution and was resuspended with lme of sterile phosphate-buffered saline (PBS) (137 mM NaCl, 3 mM KCl, 8 mM Na₂HPO₄, 1 mM KH₂PO₄, pH 7.3) before biopanning.

7.3.2 Results

The transformation efficiency of TG1 cells including pLT-2 with circular pHf1g3A-2 DNA was 10⁸/μg DNA or more, which was about 100 times as high as ligated DNA. Using an appropriate amount of supercoil pHf1g3A-2 phagemid DNA in electroporation, E. coli colonies were obtained, which were sufficiently transformed such that DVFAB-1L or DVFAB-131L has an antibody diversity of 1.3×10⁷ or 1.5×10⁹. After electroporation, 2×10⁸ or 5×10⁹ individual E. coli colonies having both phenotypes amp^{R} and tet^{R} were finally obtained from the DVFAB-1L or DVFAB-131L library. 24 E. coli colonies were randomly selected from each library, and ELISA using anti kappa light chain pAb or anti-pIII mAb was carried out to measure the ratio of E. coli clones expressing water-soluble heavy chain (V_{H}+C_{H1}) or light chain (V_{L}+C_{LK}) molecules in culture supernatant. As expected, all clones produced light chain (V_{L}+C_{LK}) molecules, 80% or more (21 among 24 clones) of the clones expressed heavy chain (V_{H}+C_{H)-g}3p fusions, so that a high level oflibrary was exhibited for E. coli clones expressing antibody fragments.

### Example 8

### Affinity-Guided Selection through DVFAB-1L

8.1 Biopanning

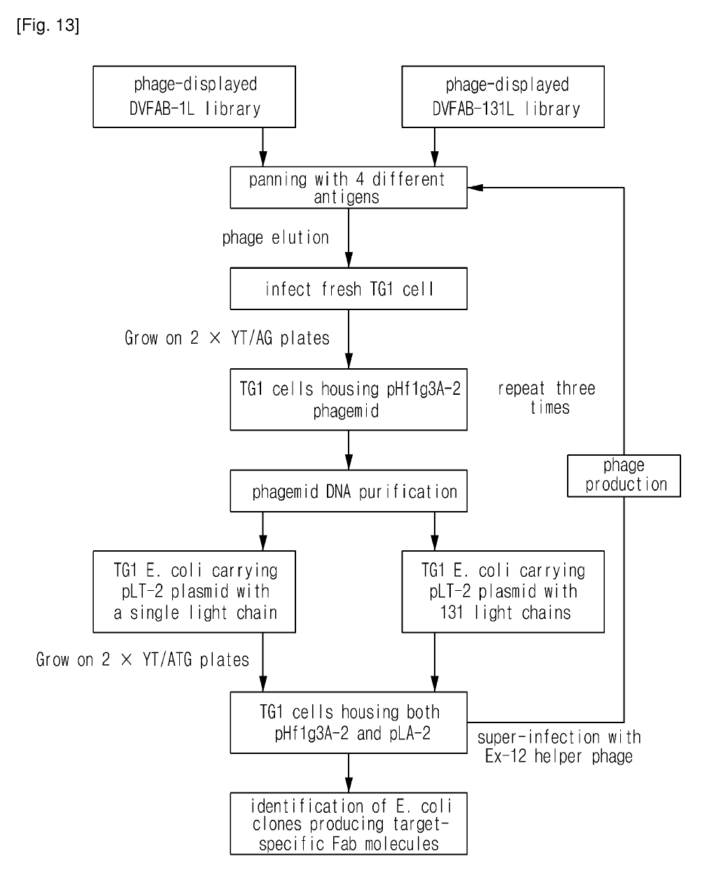

The panning procedure is as illustrated in FIG. 13 (Reference: Baek, H. J., Suk, K. H., Kim, Y. H. and Cha, S. H., (2002), An improved helper phage system for efficient isolation of specific antibody molecules in phage display, Nucleic Acids Res., 30, e18).

The MaxiSorb ELISA plate (Nunc, Denmark) was coated with 10 μg/ml fluorescein conjugated to bovine serum albumin (fluorescein-BSA) (Sigma-Aldrich), biotin-BSA (Sigma-Aldrich), bovine superoxide dismutase (bSOD) (Sigma-Aldrich), recombinant glutathione-S-transferase (GST), or L-glutamate dyhydrogenase (L-Glu) (Sigma-Aldrich) in coating buffer (0.1M NaHCO₃, pH 9.6). After the ELISA plate was culture at 4° C. overnight, ELISA wells were blocked with 3% skim milk in PBS at room temperature for 1 hour, 10¹⁰ phage from the DVFAB-1L library was added to the plate, and then the plate was cultured at 37° C. for 2 hours. The plate was washed with PBS containing 0.1% Tween 20 (PBST) eight times to remove unbound phage. Subsequently, bound phage was eluted by added 500/well buffer (0.2M glycin-HCl, pH 2.5) thereto, and was mixed with fresh TG1 cells in 2×YT medium. The TG1 cells was cultured at 27° C. overnight and were applied onto a 2×YT/ACG plate to carry out antibiotic selection. Cells were obtained from the plate by using a sterilized glass rod sterilized in fresh 2×YT medium, and phagemid DNA was isolated using the Wizard plasmid cleanup kit. Subsequently, 200 μl of electrocompetent TG1 cells containing pLT-2 plasmid encoding a single light chain were transformed with 200 ng of phagemid DNA by using the Gene Pulser. The transformed cells were applied onto a 2×YT/AT plate, and were cultured at 27° C. overnight. Next, cells were obtained from the plate, and phage was isolated with Ex-12 helper phage in 100ml of 2×YT/ATKA as described above. Biopanning was repeated three times.

Also, for the DVFAB-131L, screening was carried out using fluorescein-BSA as a target antigen in the same manner as described above, except that 10¹¹ phage was introduced in the first panning round, and TG1 cells having pLT-2 plasmid encoding 131 different light chains were used.

8.2 Target-Specific Selection

TG1 cells were superinfected from the DVFAB-1L library having Ex-12 helper phage to propagate recombinant phage. The existence of Fd (V_{H}+C_{H1})-g3p fusions and kappa light chain molecules displayed on the surface of the phage was identified by immunoblot using anti-pIII or antihuman kappa L Ab before biopanning.

Affinity-guided selection was consecutively carried out three times for fluorescein-BSA, biotin-BSA, bSOD, GST, or L-Glu. In the case of fluorescein-BSA, the number of E. coli colonies obtained after the third panning round increased by 500 times as compared to BSA, that is, a negative control antigen included in the last panning round, from which it was confirmed that recombinant phage displaying a target-specific Fab fragment was amplified. Similar results were also obtained for GST, biotin-BSA, and bSOD, but recombinant phage was amplified at a lower level than fluorescein-BSA.

Specific selection of phage using pHf1g3A-2 phagemid genome encoding target-specific Fd-ΔpIII fusions was additionally confirmed by polyclonal phage ELISA. Each recombinant phage obtained by panning with different target antigens was added into each well (5×10⁷ PFU/well) of the MaxiSorb ELISA plate coated with 10 μg/ml fluorescein-BSA, biotin-BSA, bSOD, GST, or L-Glu in coating buffer. BSA (Takara) was also included as a negative control antigen. After the plate was cultured at 37° C. for 2 hours, the plate was washed with PBST four times, and rat anti-M13 pAb (IG Therapy) was added into each well.

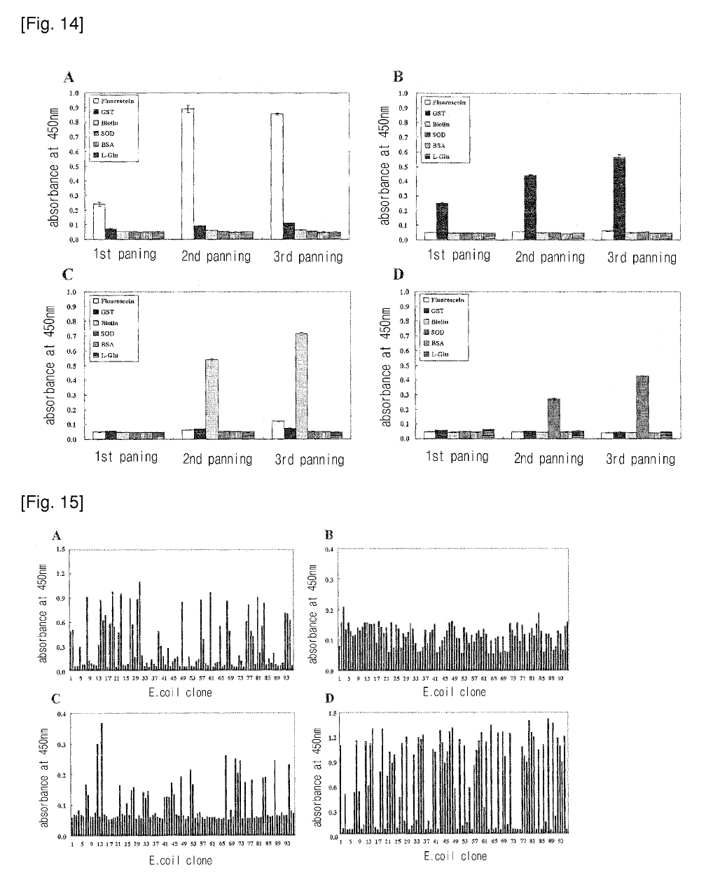

Amplification of a fluorescein-BSA (A of FIG. 14) or GST (B of FIG. 14)-specific phage antibody appeared even after the first panning round, and a biotin-BSA or bSOD-specific enrichment of phage distinctly appeared after the second panning round (C and D of FIG. 14). Production of each target-specific phage did not exhibit binding cross-reactivity with each of the five different experimented antigens, and thus binding specificity of a phage antibody was confirmed.

To identify E. coli clones expressing target-specific Fab molecules at the clone level, monoclonal ELISA was carried out. In this monoclonal ELISA, a culture supernatant of 96 independent E. coli colonies obtained after final selection for fluorescein-BSA (A of FIG. 15), GST (B of FIG. 15), biotin-BSA (C of FIG. 15), or bSOD (D of FIG. 15) was used. The frequency of positive clones producing target-specific Fab molecules varies from 30 to 70% according to antigens used for panning. It was noteworthy that GST-specific water-soluble Fab clones exhibited a very low binding signal (B of FIG. 15), as compared to the phage display antibody (B of FIG. 14) appearing in the phage ELISA.

In order to identify target antigen-specific binding of water-soluble Fab molecules, 4 to 6 E. coli clones generating a high binding signal for a target antigen in the monoclonal ELISA were selected, and ELISA was carried out using 6 different antigens. Similar to the phage ELISA in FIG. 14, water-soluble Fab molecules only reacted with their target antigen, and cross-reactivity with 5 other antigens was never observed. 6 water-soluble Fab molecules specific for GST also exhibited a very low binding signal (B of FIG. 15), and thus it was confirmed that these molecules have low affinity for the antigen or water-soluble Fab and the phage displayed antibodies may have a slightly different conformation.

### Example 9

### Analysis of Fab Clone specific for Fluorescein-BSA or bSOD-Specific

9.1 Competitive ELISA

In order to measure the binding affinity of a fluorescein-BSA or bSOD-specific Fab clone, additional competitive ELISA was carried out (References: Cha, S. H., Leung, P. S. C., Gershwin, M. E., Fletcher, M. P., Ansari, A. A. and Coppel, R. L., (1993), Combinatorial autoantibodies to dihydrolipoamide acetyltransferase, the major autoantigen of primary biliary cirrhosis, Proc. Natl. Acad. Sci., USA., 90, 2527-2531; Lee, C. V., Liang, W. C., Dennis, M. S., Eigenbrot, C., Sidhu, S. S, and Fuh, G., (2004), High-affinity human antibodies from phage-displayed synthetic Fab libraries with a single framework scaffold. J. Mol. Biol., 340, 1073-1093).

E. coli culture supernatant containing water-soluble Fab molecules was mixed with or without 10⁻⁵M to 10⁻¹²M of fluorescein or b-SOD diluted in 0.5% (w/v) in PBS and incubated at roam temperature for 2 hours. The mixture of Fab and antigen(s) was moved to the MaxiSorb ELISA plate coated with 10 μg/ml fluorescein-BSA or bSOD, and incubated with the antigen for 30 minutes. The plate was washed with PBST four times, and ELISA was carried out as described above. IC₅₀ was calculated as the concentration of solution-phage fluorescein or bSOD that inhibited 50% of Fab molecule from binding to a immobilized antigen without presence of other competitive antigens.

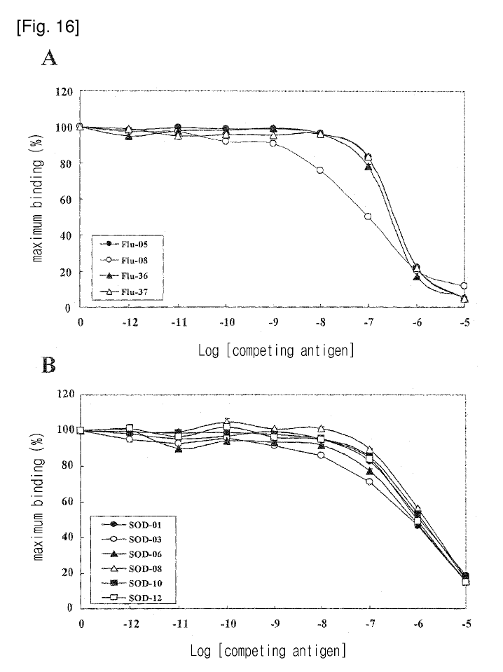

Among four fluorescein-BSA-specific clones, three clones (Flu-05, Flu-36, and Flu-37) exhibited IC₅₀=5×10⁻⁶M, and Flu-08 exhibited IC₅₀=10⁻⁷M, so that fluorescein-specific Fab clones were proven to have mid- or low-affinity for the culture (A of FIG. 16). Similarly, bSOD specificity for all the six Fab clones exhibited almost the same IC₅₀=10⁻⁶M (B of FIG. 16).

9.2 V_{H} and V_{L} DNA Sequence Analysis

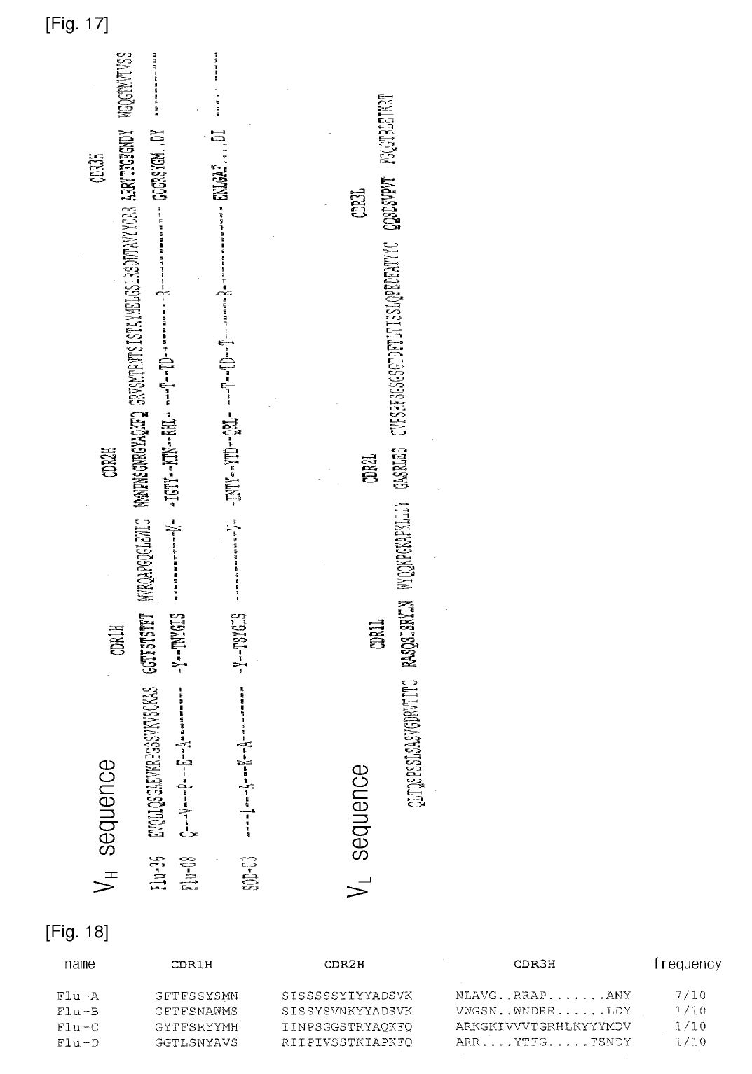

DNA sequencing was carried out to analyze the derived amino acid sequences of clones (FIG. 17). Using the Wizard plus SV minipreps kit (Promega), pHf1g3A-2 phagemid and pLT-2 plasmid were isolated from E. coli cells producing fluorescein or bSOD-specific Fab molecules. V_{H} and V_{L} genes were analyzed using two different sequencing primers complementary to pHf1g3A-2 or pLT-2 respectively, and automatic DNA sequencing (Solgent Co., South Korea) was carried out.

DNA sequencing analysis for anti-fluorescein clones proved that Flu-05, Flu-36, and Flu-37 which showed the same IC₅₀ were indeed identical. In FIG. 17, deduced amino acid sequences of two different heavy chains, Flu-36 (EMBL accession No. FM160409) and Flu-08 (EMBL accession No. FM160410), were given. Both the sequences belong to V_{H} subgroup I. DNA sequencing for six additional anti-fluorescein Fab clones was also carried out using these two V_{H} genes. In the case of six Fab clones specific to bSOD (SOD-01, SOD-03, SOD-06, SOD-08, SOD-10, and SOD-12), it was found that they are all identical in the V_{H} amino acid sequences (EMBL accession No. FM160411) belonging to the V_{H} subgroup I (FIG. 17). From such results, it was confirmed that there are a few target-specific heavy chains in the heavy chain repertoire of the DVFAB-1L library. The amino acid sequence of single V_{L} kappa (EMBL accession NO. FM160412) used in the DVFAB-1L library is given in FIG. 17.

### Example 10

### Isolation of Fluorescein-BSA-Specific Fab Clone from DVFAB-131L Library

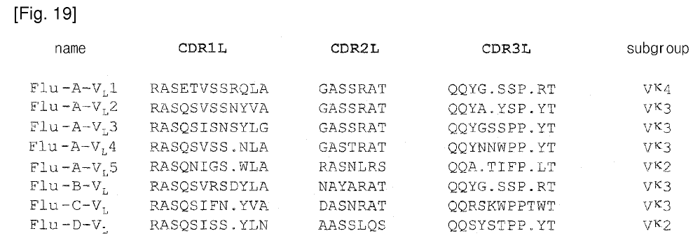

The DVFAB-131L library having a combinatorial Fab repertoire that is 131 times as large as the DVFAB-1L was produced by a random combination of 131 light chains having the same heavy chain repertoire. In producing the library, supercoil-shaped pHf1g3A-2 DNA was used, and about 5×10⁹ transformed E. coli colonies can be obtained within a day. Since the haptenic of fluorescein is helpful to understand the antibody repertoire of a library, the produced library was screened with fluorescein-BSA. After three rounds of panning, monoclonal ELISA was carried out (FIG. 15) to identify E. coli clones producing an anti-fluorescein Fab fragment. A total of 384 E. coli clones were analyzed. The frequency of E. coli clones producing water-soluble Fab molecules against fluorescein was about 4%, which was significantly lower than that for DVFAB-1L. This is because amplified heavy chain genes were randomly reshuffled with independent 131 light chains through panning after each round of panning. Among positive Fab clones, 10 clones exhibiting high binding reactivity to fluorescein but not exhibiting cross-reactivity to irrelevant antigens were selected, and DNA sequences of V_{H} and V_{L} genes of the Fab clones were determined (FIGS. 18 and 19). Four different V_{H} genes named Flu-A (EMBL accession No. FM160413), Flu-B (EMBL accession No. FM160414), Flu-C (EMBL accession No. FM160415), and Flu-D (EMBL accession No. FM160416) were identified among ten Fab clones (FIG. 18). Flu-A V_{H} gene was used by seven Fab clones, and the Flu-B V_{H}, Flu-C V_{H}, Flu-D V_{H} genes were represented by each of rest three Fab clones. Through analysis of deduced amino acid sequences, it was confirmed that Flu-A and Flu-B V_{H} genes belong to V_{H} subgroup III, and other two genes, that is, Flu-C and Flu-D V_{H} genes, belong to V_{H} subgroup I. Eight different V_{L} genes were used as light chains by the ten Fab clones (FIG. 19). The Fab clone having Flu-A V_{H} was paired with five different light chains called Flu-A-V_{L}1 (EMBL accession No. FM160417), Flu-A-V_{L}2 (EMBL accession No. FM160418), Flu-A-V_{L}3 (EMBL accession No. FM160419), Flu-A-V_{L}4 (EMBL accession No. FM160420), and Flu-A-V_{L}5 (EMBL accession No. FM160421) respectively, indicating that Flu-A V_{H} has the highest light chain promiscuity. Contrarily, each heavy chain Flu-B, Flu-C, or Flu-D was paired with Flu-B-V_{L} (EMBL accession No. FM160422), Flu-C-V_{L} (EMBL accession No. FM160423), or Flu-D-V_{L} (EMBL accession No. FM160424). All the clones had a K_{D} value of approximately 10⁻⁶, as D measured by IC₅₀.

DVS-II technology can be used as a tool useful for producing a combinatorial phage display Fab library with high diversity. Further, it can be practically used to select a desired antibody clone through panning in consideration of flexibility of light chains in the antigen-antibody binding reaction of an antibody, and can be very effectively utilized to produce a human antibody by manipulating at least a monoclonal antibody of rodent origin through guided-selection or chain shuffling.

In all aspects including vector stability, the amount of expression of water-soluble Fab molecules, the titer of produced recombinant phage, the amount of antibody molecules displayed on the surface of phage, and a selection function of recombinant phage displaying an antigen-specific antibody, etc., DVS-II may be comparable with the existing phage display system using a single phagemid vector.

The usefulness of an antibody library is directly related to the number of clones constituting the antibody library, and thus it can be inferred that the more clones in a library, the larger the antigen-binding specificity of the library. Further, a possibility to obtain a useful antibody binding to a specific antigen with high affinity may increase, and thus DVS-II can be very effectively used for combinatorial Fab fragment library production.

Also, in consideration that both light and heavy chain fragments must be expressed by one vector in conventional single vector system, the dual-vector system of the present invention can prevent degradation of antibody gene diversity due to restriction enzymes used for antibody cloning in combinatorial Fab fragment library production as much as possible because it includes independent two vectors.

In addition, DVS-II can select target molecule-specific heavy chain gene to be paired with specific monoclonal light chain gene, and can be directly applied to chain shuffling or guided-selection used for transforming monoclonal antibody gene of rodent origin into antibody gene of human origin. With regard to this, the most important advantage of DVS-II is that if once a superior heavy chain gene library is produced with pHf1g3A-2, this library can be used to secure human heavy chain gene binding to all light chain genes of rodent origin and exhibiting binding specificity for a specific antigen.

The most important advantage of the DVS-II system of the present invention is a combinatorial Fab diversity of 10¹¹ can be quickly and accurately obtained by a random combination of 131 light chains in pLT-2 plasmid, and can be easily applied to humanization of non-human mAbs. Once a reliable heavy chain repertoire is formed by DVS-II, target-specific human heavy chains can be obtained by combining the repertoire with any light chain of non-human mAb without constructing heavy chain libraries for all cases.

1-10. (canceled)

11. A method for producing transformants comprising the steps of:
(1) producing a first vector by liqatinq a pBR322 plasmid and a light chain of a human antibody;
(2) producing a second vector by generating a first DNA fragment by subjecting a pBAD/gIII plasmid to a PCR reaction with a primer set of Sequence Nos. 3 and 4; generating a second DNA fragment by subjecting a pCDFDuet-1 plasmid to a PCR reaction with a primer set of Sequence Nos. 5 and 6; liqatinq the first DNA fragment and the second DNA fragment; and ligating a heavy chain of a human antibody;
(3) transforming host cells by using the first vector in the step (1); and
(4) transforming the host cells transformed in the step (3), by using the second vector in the step (2).

12. A method for producing transformants comprising the steps of:
(1) producing a first vector by ligating a pBR322 plasmid and a light chain of a human antibody;
(2) producing a second vector by generating a first DNA fragment by subjecting a pBAD/qIII plasmid to a PCR reaction with a primer set of Sequence Nos. 3 and 4; generating a second DNA fragment by subjecting a pCDFDuet-1 plasmid to a PCR reaction with a primer set of Sequence Nos. 5 and 6; ligating the first DNA fragment and the second DNA fragment; and ligating a heavy chain of a human antibody;
(3) transforming host cells by using the second vector in the step (2); and
(4) transforming the host cells transformed in the step (3), by using the first vector in the step (1).

13. (canceled)

14. The method of expressing a human antibody Fab fragment gene by using the method as claimed in claim 11 or 12.

15. The method of producing a recombinant phage displaying a human antibody Fab fragment by using the method as claimed in claim 11 or 12.

16. The method of selecting a recombinant phage having a target molecule-specific VH+CH1 antibody gene phagemid genome by using the method as claimed in claim 11 or 12.

17-25. (canceled)


SEQUENCE LISTING (14 sequences)

SEQ ID NO: 1 - DNA, 34 bp, Artificial Sequence
  Sense primer
gggctgcagacgcggcctttttacggtggttcct

SEQ ID NO: 2 - DNA, 31 bp, Artificial Sequence
  Anti-sense primer
gggcaattgccgcgcacatttccccgaaaag

SEQ ID NO: 3 - DNA, 32 bp, Artificial Sequence
  Sense primer
gggatcgattcaattgtctgattcgttaccaa

SEQ ID NO: 4 - DNA, 30 bp, Artificial Sequence
  Anti-sense primer
gggactagttcagtggaacgaaaactcacg

SEQ ID NO: 5 - DNA, 29 bp, Artificial Sequence
  Sense primer
gggatcgatatagctagctcactcggtcg

SEQ ID NO: 6 - DNA, 30 bp, Artificial Sequence
  Anti-sense primer
gggactagtgcactgaaatctagagcggaa

SEQ ID NO: 7 - DNA, 47 bp, Artificial Sequence
  Sense primer
gggccatgggatttaggtgacactataggatctcgatcccgcgaaat

SEQ ID NO: 8 - DNA, 34 bp, Artificial Sequence
  Anti-sense primer
gggctcgagttatcaacactctcccctgttgctc

SEQ ID NO: 9 - DNA, 35 bp, Artificial Sequence
  VLkappaaSal
ggggtcgacatggacatccagatgacccagtctcc

SEQ ID NO: 10 - DNA, 35 bp, Artificial Sequence
  JkappaSac
gggcggcggatacgtttgathtccasyttggtccc

SEQ ID NO: 11 - DNA, 34 bp, Artificial Sequence; identical to SEQ ID NO: 1
  Sense primer

SEQ ID NO: 12 - DNA, 31 bp, Artificial Sequence; identical to SEQ ID NO: 2
  Anti-sense primer

SEQ ID NO: 13 - DNA, 32 bp, Artificial Sequence; identical to SEQ ID NO: 3
  Sense primer

SEQ ID NO: 14 - DNA, 30 bp, Artificial Sequence; identical to SEQ ID NO: 4
  Anti-sense primer